(19) United States
(12) Patent Application Publication
(10) Pub. No.: US 2005/0250814 A1
(43) Pub. Date: Nov. 10, 2005

(54) Piperidinyl-alpha-aminoamide modulators of chemokine receptor activity

(76) Inventors: Cangyou Zhou, Plainsboro, NJ (US); Lihu Yang, Edison, NJ (US)

Correspondence Address: MERCK AND CO., INC, P O BOX 2000, RAHWAY, NJ, 07065-0907, US

(21) Appl. No.: 10/528,293

(22) PCT Filed: Oct. 24, 2003
(86) PCT No.: PCT/US03/33980

§ 371 Date: Mar. 16, 2005

Related U.S. Application Data
(60) Provisional application No. 60/422,355, filed on Oct. 30, 2002.

(51) Int. Cl. A61K 31/454; C07D 43/02

(52) U.S. Cl. 514/323; 514/326; 546/200; 546/208

(57) ABSTRACT

The present invention is directed to compounds of the formula I: (wherein R₁, R₂, R₃, R₄, R₅, R₆, W, X, Z, m and n are defined herein) which are useful as modulators of chemokine receptor activity. In particular, these compounds are useful as modulators of the chemokine receptor CCR-2.

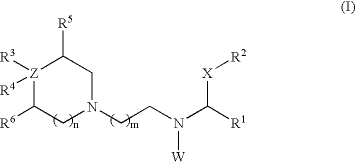

### BACKGROUND OF THE INVENTION

The chemokines are a family of small (70-120 amino acids), proinflammatory cytokines, with potent chemotactic activities. Chemokines are chemotactic cytokines that are released by a wide variety of cells to attract various cells, such as monocytes, macrophages, T cells, eosinophils, basophils and neutrophils to sites of inflammation (reviewed in Schall, Cytokine, 37 165-183 (1991) and Murphy, Rev. Immun., 12, 593-633 (1994)). These molecules were originally defined by four conserved cysteines and divided into two subfamilies based on the arrangement of the first cysteine pair. In the CXC-chemokine family, which includes IL-8, GROα, NAP-2 and IP-10, these two cysteines are separated by a single amino acid, while in the CC-chemokine family, which includes RANTES, MCP-1, MCP-2, MCP-3, MIP-1α, MIP-1β and eotaxin, these two residues are adjacent.

The α-chemokines, such as interleukin-8 (IL-8), neutrophil-activating protein-2 (NAP-2) and melanoma growth stimulatory activity protein (MGSA) are chemotactic primarily for neutrophils, whereas β-chemokines, such as RANTES, MIP-1α, MIP-1β, monocyte chemotactic protein-1 (MCP-1), MCP-2, MCP-3 and eotaxin are chemotactic for macrophages, monocytes, T-cells, eosinophils and basophils (Deng, et al., Nature, 381, 661-666 (1996)).

The chemokines are secreted by a wide variety of cell types and bind to specific G-protein coupled receptors (GPCRs) (reviewed in Horuk, Trends Pharm. Sci ., 15, 159-165 (1994)) present on leukocytes and other cells. These chemokine receptors form a sub-family of GPCRs, which, at present, consists of fifteen characterized members and a number of orphans. Unlike receptors for promiscuous chemoattractants such as C5a, fMLP, PAF, and LTB4, chemokine receptors are more selectively expressed on subsets of leukocytes. Thus, generation of specific chemokines provides a mechanism for recruitment of particular leukocyte subsets.

On binding their cognate ligands, chemokine receptors transduce an intracellular signal though the associated trimeric G protein, resulting in a rapid increase in intracellular calcium concentration. There are at least seven human chemokine receptors that bind or respond to β-chemokines with the following characteristic pattern: CCR-1 (or “CKR-1” or “CC-CKR-1”) [MIP-1α, MIP-1β, MCP-3, RANTES] (Ben-Barruch, et al., J. Biol. Chem., 270 22123-22128 (1995); Beote, et al, Cell, 72, 415-425 (1993)); CCR-2A and CCR-2B (or “CKR-2A”/“CKR-2A” or “CC-CKR-2A”/“CC-CKR-2A”) [MCP-1, MCP-2, MCP-3, MCP4]; CCR-3 (or “CKR-3” or “CC-CKR-3”) [Eotaxin, Eotaxin 2, RANTES, MCP-2, MCP-3] (Rollins, et al., Blood, 90, 908-928 (1997)); CCR4 (or “CKR4” or “CC-CKR-4”) [MIP-1α, RANTES, MCP-1] (Rollins, et al., Blood, 90, 908-928 (1997)); CCR-5 (or “CKR-5” or “CC-CKR-5”) [MIP-1α, RANTES, MIP-1β] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”) among other chemokines.

Chemokine receptors, such as CCR-1, CCR-2, CCR-2A, CCR-2B, CCR-3, CCR-4, CCR-5, CXCR-3, CXCR-4, have been implicated as being important mediators of inflammatory and immunoregulatory disorders and diseases, including asthma, rhinitis and allergic diseases, as well as autoimmune pathologies such as rheumatoid arthritis and atherosclerosis. Humans who are homozygous for the 32-basepair deletion in the CCR-5 gene appear to have less susceptibility to rheumatoid artritis (Gomez, et al., Arthritis & Rheumatism, 42, 989-992 (1999)). A review of the role of eosinophils in allergic inflammation is provided by Kita, H., et al., J. Exp. Med. 183, 2421-2426 (1996). A general review of the role of chemokines in allergic inflammation is provided by Lustger, A. D., New England J. Med., 338(7), 426-445 (1998).

A subset of chemokines are potent chemoattractants for monocytes and macrophages. The best characterized of these is MCP-1 (monocyte chemoattractant protein-1), whose primary receptor is CCR2. MCP-1 is produced in a variety of cell types in response to inflammatory stimuli in various species, including rodents and humans, and stimulates chemotaxis in monocytes and a subset of lymphocytes. In particular, MCP-1 production correlates with monocyte and macrophage infiltration at inflammatory sites. Deletion of either MCP-1 or CCR2 by homologous recombination in mice results in marked attenuation of monocyte recruitment in response to thioglycollate injection and Listeria monocytogenes infection (Lu et al., J. Exp. Med 187:601-608 (1998); Kurihara et al. J. Exp. Med. 186: 1757-1762 (1997); Boring et al. J. Clin. Invest. 100:2552-2561 (1997); Kuziel et al. Proc. Natl. Acad. Sci. 94:12053-12058 (1997)). Furthermore, these animals show reduced monocyte infiltration into granulomatous lesions induced by the injection of schistosomal or mycobacterial antigens (Boring et al. J. Clin. Invest. 100:2552-2561 (1997); Warmington et al. Am 3. Path. 154:1407-1416 (1999)). These data suggest that MCP-1-induced CCR2 activation plays a major role in monocyte recruitment to inflammatory sites, and that antagonism of this activity will produce a sufficient suppression of the immune response to produce therapeutic benefits in immunoinflammatory and autoimmune diseases

Accordingly, agents which modulate chemokine receptors such as the CCR-2 receptor would be useful in such disorders and diseases.

In addition, the recruitment of monocytes to inflammatory lesions in the vascular wall is a major component of the pathogenesis of atherogenic plaque formation. MCP-1 is produced and secreted by endothelial cells and intimal smooth muscle cells after injury to the vascular wall in hypercholesterolemic conditions. Monocytes recruited to the site of injury infiltrate the vascular wall and differentiate to foam cells in response to the released MCP-1. Several groups have now demonstrated that aortic lesion size, macrophage content and necrosis are attenuated in MCP-1−/− or CCR2−/− mice backcrossed to APO-E−/−, LDL-R−/− or Apo B transgenic mice maintained on high fat diets (Boring et al. Nature 394:894-897 (1998); Gosling et al. J. Clin. Invest. 103:773-778 (1999)). Thus, CCR2 antagonists may inhibit atherosclerotic lesion formation and pathological progression by impairing monocyte recruitment and differentiation in the arterial wall.

### SUMMARY OF THE INVENTION

The present invention is further directed to compounds which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

### DETAILED DESCRIPTION OF THE INVENTION

The present invention is directed to compounds of the formula I: 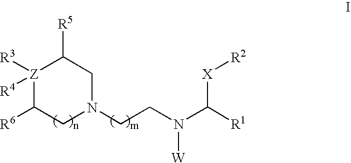
wherein:
X is selected from the group consisting of:
   —NR¹⁰—, —O—, —CH₂O—, CONR¹⁰—, —NR¹⁰CO—, —CO₂—, —OCO—, —CH₂(NR¹⁰)CO—, —N(COR¹⁰)—, —CH₂N(COR¹⁰)—, phenyl, and C₃₋₆ cycloalkyl,
   where R¹⁰ is independently selected from: hydrogen, C₁₋₆ alkyl, benzyl, phenyl, and C₁₋₆ alkyl-C₃₋₆ cycloalkyl,
      which is unsubstituted or substituted with 1-3 substituents where the substituents are independently selected from: halo, C₁₋₃alkyl,
      C₁₋₃alkoxy and trifluoromethyl;
W is selected from:
   hydrogen and C₁₋₆alkyl, which is unsubstituted or substituted with 1-3 substituents where the substituents are independently selected from: halo, C₁₋₃alkoxy and trifluoromethyl;
Z is selected from:
   C, N, and —O—, wherein when Z is N, then R⁴ is absent, and when W is —O—, then both R³ and R⁴ are absent;
n is an integer selected from 0, 1, 2, 3 and 4;
n is an integer selected from 1, 2, 3 and 4;
R¹ is selected from:
   hydrogen, —C₀₋₆alkyl-, —(C₀₋₆alkyl)-alkenyl-,
   —(C₀₋₆alkyl)-C₃₋₆cycloalkyl, —(C₀₋₆alkyl)-phenyl, and —(C₀₋₆alkyl)-heterocycle,
   where the alkyl is unsubstituted or substituted with 1-7 substituents where the substituents are independently selected from:
   (a) halo,
   (b) hydroxy,
   (c) —O—C₁₋₃alkyl,
   (d) trifluoromethyl, and
   (e) —C₁₋₃alkyl,
and where the phenyl and the heterocycle is unsubstituted or substituted with 1-5 substituents where the substituents are independently selected from:
   (a) halo,
   (b) hydroxy; alkoxy
   (c) amino; acylamino;sulfonylamino; alkoxycarbonylamino
   (d) carboxylic acid; carbamide; sulfonamide
or wherein W and R¹ may be joined together to form a ring by a group selected from:
   —(C₁₋₆alkyl)-, —C₀₋₆alkyl-Y—(C₁₋₆alkyl)-, and
   —(C₀₋₆alkyl)-Y—(C₀₋₆alkyl)-(C₃₋₇cycloalkyl)-(C₀₋₆alkyl),
      where Y is selected from:
      a single bond, —O—, —S—, —SO—, —SO₂—, and —NR¹⁰—,
      and where the alkyl and the cycloalkyl are unsubstituted or substituted with 1-7 substituents where the substituents are independently selected from:
      (a) halo,
      (b) hydroxy,
      (c) —O—C₁₋₃alkyl, and
      (d) trifluoromethyl,
      (e) C₁₋₃alkyl,
      (f) —O—C₁₋₃alkyl,
      (g) —CO₂R⁹, wherein R⁹ is independently selected from: hydrogen, C₁₋₆ alkyl, C₅₋₆ cycloalkyl, benzyl or phenyl, which is unsubstituted or substituted with 1-3 substituents where the substituents are independently selected from: halo, C₁₋₃alkyl, C₁₋₃alkoxy and trifluoromethyl,
      (h) —CN,
      (i) —NR⁹R¹⁰,
      (j) —NR⁹COR¹⁰,
      (k) —NR⁹SO₂R¹⁰, and
      (l) —CONR⁹R¹⁰;
R² is selected from:
   (C₀₋₆alkyl)-phenyl and (C₀₋₆alkyl)-heterocycle,
      where the alkyl is unsubstituted or substituted with 1-7 substituents where the substituents are independently selected from:
      (a) halo,
      (b) hydroxy,
      (c) —O—C₁₋₃alkyl,
      (d) trifluoromethyl, and
      (e) —C₁₋₃alkyl,
   and where the phenyl and the heterocycle is unsubstituted or substituted with 1-5 substituents where the substituents are independently selected from:
      (a) halo,
      (b) trifluoromethyl,
      (c) trifluoromethoxy,
      (d) hydroxy,
      (e) C₁₋₆alkyl,
      (f) C₃₋₇cycloalkyl,
      (g) —O—C₁₋₆alkyl,
      (h) —O—C₃₋₇cycloalkyl,
      (i) —SCF₃,
      (j) —S—C₁₋₆alkyl,
      (k) —SO₂—C₁₋₆alkyl,
      (l) phenyl,
      (m) heterocycle,
      (n) —CO₂R⁹,
      (o) —CN,
      (p) —NR⁹R¹⁰,
      (q) —NR⁹—SO₂—R¹⁰,
      (r) —SO₂—NR⁹R¹⁰, and
      (s) —CONR⁹R¹⁰;
R³ is —(C₀₋₆alkyl)-phenyl,
   where the alkyl is unsubstituted or substituted with 1-5 substituents where the substituents are independently selected from:
   (a) halo,
   (b) hydroxy,
   (c) —O—C₁₋₃alkyl, and
   (d) trifluoromethyl,
and where the phenyl is unsubstituted or substituted with 1-5 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —CO₂R⁹,
   (g) —CN,
   (h) —NR⁹R¹⁰, and
   (i) —CONR⁹R¹⁰;
R⁴ is selected from:
   (a) hydrogen,
   (b) hydroxy,
   (c) C₁₋₆alkyl,
   (d) C₁₋₆alkyl-hydroxy,
   (e) —O—C₁₋₃alkyl,
   (f) —CO₂R⁹,
   (g) —CONR⁹R¹⁰, and
   (h) —CN;
or where R³ and R⁴ may be joined together to form a ring which is selected from:
   (a) 1H-indene,
   (b) 2,3-dihydro-1H-indene,
   (c) 2,3-dihydro-benzofuran,
   (d) 1,3-dihydro-isobenzofuran,
   (e) 2,3-dihydro-benzothiofuran, and
   (f) 1,3-dihydro-isobenzothiofuran,
or where R³ and R⁵ or R⁴ and R⁶ may be joined together to form a ring which is phenyl,
   wherein the ring is unsubstituted or substituted with 1-7 substituents where the substituents are independently selected from:
      (a) halo,
      (b) trifluoromethyl,
      (c) hydroxy,
      (d) C₁₋₃alkyl,
      (e) —O—C₁₋₃alkyl,
      (f) —CO₂R⁹,
      (g) —CN,
      (h) —NR⁹R¹⁰, and
      (i) —CONR⁹R¹⁰;
R⁵ and R⁶ are independently selected from:
   (a) hydrogen,
   (b) hydroxy,
   (c) C₁₋₆alkyl,
   (d) C₁₋₆alkyl-hydroxy,
   (e) —O—C₁₋₃alkyl,
   (f) oxo, and
   (g) halo;
and pharmaceutically acceptable salts thereof and individual diastereomers thereof.

Another embodiment of the present invention is directed to compounds of formula Ia: 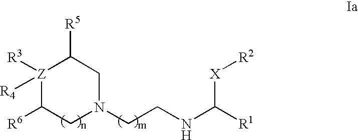
wherein R¹, R², R³, R³, n, m, X and Z are defined herein;
and pharmaceutically acceptable salts and individual diastereomers thereof.

Another embodiment of the present invention is directed to compounds of formula Ib: 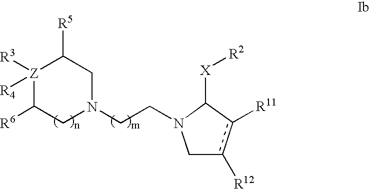
wherein the dashed line represents a single or a double bond and R², R³, R⁴, n, X and Z are defined herein,
and R¹¹ is selected from:
   (a) hydrogen
   (b) C₁₋₆alkyl
   (c) hydroxy,
   (d) —O—C₁₋₃alkyl
   (e) -Phenyl and heterocycle,
   (f) —CO₂R⁹,
   (g) —CN,
   (h) —NR⁹R¹⁰, and
   (i) —CONR⁹R¹⁰;
R¹² is selected from:
   (a) hydrogen,
   (b) hydroxy,
   (c) C₁₋₆alkyl,
   (d) C₁₋₆alkyl-hydroxy,
   (e) —O—C₁₋₃alkyl,
   (f) —CO₂R⁹,
   (g) —CONR⁹R¹⁰, and
   (h) —CN;
or where R¹¹ and R¹² may be joined together to form a ring which is selected from:
   (a) benzene,
   (b) furan,
   (c) thiophene,
   (d) thiazole,
   (e) C₃₋₆cycloalkyl
wherein the ring is unsubstituted or substituted with 1-7 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —CO₂R⁹,
   (g) —CN,
   (h) —NR⁹R¹⁰, and
   (i) —CONR⁹R¹⁰;
and pharmaceutically acceptable salts and individual diastereomers thereof.

Another embodiment of the present invention is directed to compounds of formula Id: 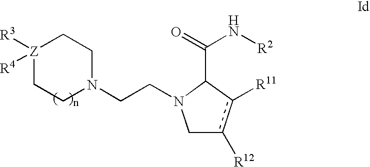
wherein R², R³, R⁴, R¹¹, R¹², n and Z are defined herein;
and pharmaceutically acceptable salts and individual diastereomers thereof.

Another embodiment of the present invention is directed to compounds of formula Ie: 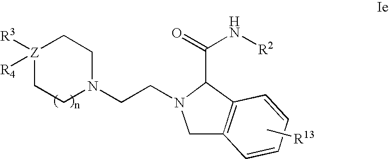
wherein the dashed line represents a single or a double bond and R², R³, R⁴, n and X are defined herein,
and wherein R¹² is independently selected from:
   (a) hydrogen,
   (b) halo,
   (c) trifluoromethyl,
   (d) fused C₁₋₃cycloalkyl
   (e) C₁₋₃alkyl,
   (f) —O—C₁₋₃alkyl,
   (g) —CO₂H,
   (h) —CO₂C₁₋₃alkyl, and
   (i) —CN;
and pharmaceutically acceptable salts and individual diastereomers thereof.

Another embodiment of the present invention is directed to compounds of formula If: 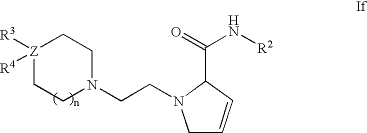
wherein R², R³, R⁴, and n are defined herein,
and pharmaceutically acceptable salts and individual diastereomers thereof.

Another embodiment of the present invention is directed to compounds of the formula II: 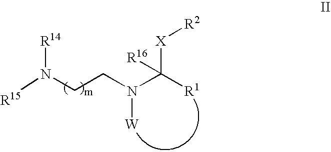
wherein R¹, R², m, W and X are defined herein,
and wherein R¹⁴, R¹⁵, R¹⁶ are independently selected from:
   (a) hydrogen,
   (b) —C₁₋₆alkyl
   (c) —C₁₋₆cycloalkyl
   (d) —C₁₋₆alkyl-phenyl
   (e) —C₁₋₆alkyl-heterocylcle
   (f) —C₁₋₆alkyl-C₃₋₆cycloalkyl
   (g) —C₁₋₆alkyl O—C₁₋₆alkyl,
and pharmaceutically acceptable salts and individual diastereomers thereof.

Another embodiment of the present invention is directed to compounds of formula IIa: 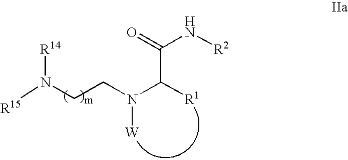
wherein R¹, R², R¹⁴, R¹⁵, m, W and X are defined herein,
and pharmaceutically acceptable salts and individual diastereomers thereof.

Another embodiment of the present invention is directed to compounds of formula IIb: 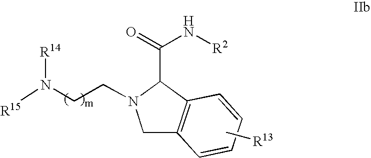
wherein R¹, R², R¹³, R¹⁴, R¹⁵, m, W and X are defined herein,
and pharmaceutically acceptable salts and individual diastereomers thereof.

Another embodiment of the present invention is directed to compounds of formula IIc: 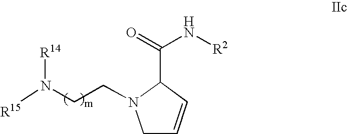
wherein R¹, R², R¹⁴, R¹⁵, m, W and X are defined herein,
and pharmaceutically acceptable salts and individual diastereomers thereof.

In the present invention it is most preferred that W is hydrogen or —CH₂—.

In the present invention it is most preferred that X is —CONH—, phenyl or heterocycle.

In the present invention it is most preferred that Z is —C— or —N—

In the present invention it is most preferred that n is 0 and 1.

In the present invention it is most preferred that m is 1.

In the present invention it is preferred that heterocycle is selected from:
furanyl, imidazolyl, oxadiazolyl, oxazolyl, pyrazolyl, pyrazinyl, pyridyl, pyridazinyl, pyrimidyl, pyrrolyl, thiadiazolyl, thiazolyl, thienyl, and triazolyl, and N-oxides thereof.

In the present invention it is preferred that R¹ is selected from:
—C₁₋₆alkyl, —C₀₋₆alkyl-O—C₁₋₆alkyl-, —C₀₋₆alkyl-S—C₁₋₆alkyl-, and
—(C₀₋₆alkyl)-(C₃₋₇cycloalkyl)-(C₀₋₆alkyl),
   where the alkyl and the cycloalkyl are unsubstituted or substituted with 1-7 substituents where the substituents are independently selected from:
   (a) halo,
   (b) hydroxy,
   (c) —O—C₁₋₃alkyl,
   (d) trifluoromethyl,
   (f) C₁₋₃alkyl,
   (g) —O—C₁₋₃alkyl,
   (h) —CO₂R⁹, wherein R⁹ is independently selected from: hydrogen, C₁₋₆ alkyl, C₅₋₆ cycloalkyl, benzyl or phenyl, which is unsubstituted or substituted with 1-3 substituents where the substituents are independently selected from; halo, C₁₋₃alkyl, C₁₋₃alkoxy and trifluoromethyl,
   (i) —CN,
   (j) —NR⁹R¹⁰, and
   (k) —CONR⁹R¹⁰.

In the present invention it is more preferred that R¹ is selected from:
(1) —C₁₋₆alkyl, which is unsubstituted or substituted with 1-6 substituents where the substituents are independently selected from:
   (a) halo,
   (b) hydroxy,
   (c) —O—C₁₋₃alkyl, and
   (d) trifluoromethyl,
(2) —CO₆alkyl-O—C₁₋₆alkyl-, which is unsubstituted or substituted with 1-6 substituents where the substituents are independently selected from:
   (a) halo, and
   (b) trifluoromethyl,
(3) —C₀₋₆alkyl-S—C₁₋₆alkyl-, which is unsubstituted or substituted with 1-6 substituents where the substituents are independently selected from:
   (a) halo, and
   (b) trifluoromethyl,
(4) —(C₃₋₅cycloalkyl)-(C₀₋₆alkyl), which is unsubstituted or substituted with 1-7 substituents where the substituents are independently selected from:
   (a) halo,
   (b) hydroxy,
   (c) —O—C₁₋₃alkyl, and
   (d) trifluoromethyl.

In the present invention it is even more preferred that R¹ is selected from:
(1) —CH₃,
(2) —CH₂CH₃,
(3) —CH(CH₃)₂,
(4) —CH₂CH₂CH₃,
(5) —CH₂CH(CH₃)₂,
(6) -cyclopropyl,
(7) -cyclobutyl,
(8) -cyclopentyl,
(9) —CH₂-cyclopropyl,
(10) —CH₂-cyclobutyl,
(11) —CH₂-cyclopentyl,
(12) —CH₂OH,
(13) —C(CH₃)₂(OH),
(14) —C(CH₂OH)(CH₃)₂,
(15) —(OH)cyclobutyl,
(16) —(OH)cyclopentyl,
(17) —C(CH₃)₂(NHCOCH₃).
(18) —C(CO₂H)(CH₃)₂,
(19) —O—CH₃,
(20) —O-cyclopentyl,
(21) —O—CH(CH₃)₂,
(22) —S—CH₃,
(23) —S—CF₃,
(24) —SO₂—CH₃,
(25) —S—CH(CH₃)₂,
(26) —SO₂—CH(CH₃)₂, and
(27) —NH—SO₂—CH₃.

In the present invention it is preferred that R² is selected from
—(C₀₋₄alkyl)-phenyl and —(C₀₋₄alkyl)-heterocycle,
   where heterocycle is selected from:
      furanyl, imidazolyl, oxadiazolyl, oxazolyl, pyrazolyl, pyrazinyl, pyridyl, pyridazinyl, pyrimidyl, pyrrolyl, thiadiazolyl, thiazolyl, thienyl, and triazolyl, and N-oxides thereof,
   where the alkyl is unsubstituted or substituted with 1-7 substituents where the substituents are independently selected from:
      (a) halo,
      (b) hydroxy,
      (c) —O—C₁₋₃alkyl, and
      (d) trifluoromethyl,
   and where the phenyl or heterocycle is unsubstituted or substituted with 1-5 substituents where the substituents are independently selected from:
      (a) halo,
      (b) trifluoromethyl,
      (c) trifluoromethoxy,
      (d) hydroxy,
      (e) C₁₋₃alkyl,
      (f) —O—C₁₋₃alkyl,
      (g) —CO₂R⁹,
      (h) —S—C₁₋₃alkyl,
      (i) —SO₂—C₁₋₃alkyl,
      (j) —SCF₃,
      (k) —CO₂R⁹,
      (l) —NR⁹R¹⁰,
      (m) —NR⁹—SO₂R¹⁰,
      (n) —SO₂—NR⁹R¹⁰, and
      (o) —CONR⁹R¹⁰.

In the present invention it is more preferred that R² is selected from
—(C₀₋₄alkyl)-phenyl and —(C₀₋₄alkyl)-heterocycle,
where heterocycle is selected from: pyridyl, pyridazinyl, and N-oxides thereof,
where the alkyl is unsubstituted or substituted with 1-7 substituents where the substituents are independently selected from:
   (a) halo,
   (b) hydroxy,
   (c) —O—C₁₋₃alkyl, and
   (d) trifluoromethyl,
and where the phenyl or heterocycle is unsubstituted or substituted with 1-3 substituents where the substituents are independently selected from:
   (a) halo,
   (b) trifluoromethyl,
   (c) trifluoromethoxy,
   (d) hydroxy,
   (e) C₁₋₃alkyl,
   (f) —O—C₁₋₃alkyl,
   (g) —CO₂—C₁₋₃alkyl,
   (h) —CO₂H,
   (i) —S—C₁₋₃alkyl,
   (j) —SO₂—C₁₋₃alkyl,
   (k) —SCF₃,
   (l) —NH₂,
   (m) —NH—SO₂—C₁₋₃alkyl, and
   (n) —SO₂—NH₂.

In the present invention it is even more preferred that R² is selected from —CH₂-phenyl and —CH₂-heterocycle,
where heterocycle is selected from: pyridyl, pyridazinyl, and N-oxides thereof,
and where the phenyl or heterocycle is unsubstituted or substituted with 1-3 substituents where the substituents are independently selected from:
   (a) halo,
   (b) trifluoromethyl,
   (c) trifluoromethoxy,
   (d) hydroxy,
   (e) C₁₋₃alkyl,
   (f) —O—C₁₋₃alkyl,
   (g) —CO₂—C₁₋₃alkyl,
   (h) —CO₂H,
   (i) —S—C₁₋₃alkyl,
   (j) —SO₂—C₁₋₃alkyl,
   (k) —SCF₃,
   (l) —NH₂,
   (m) —NH—SO₂—C₁₋₃alkyl, and
   (n) —SO₂—NH₂.

In the present invention it is still more preferred that R² is selected from:
(1) —CH₂-(phenyl),
(2) —CH₂-(4-bromophenyl),
(3) —CH₂-(3-chlorophenyl),
(4) —CH₂-(3,5-difluorophenyl),
(5) —CH₂-((2-trifluoromethyl)phenyl),
(6) —CH₂-((3-trifluoromethyl)phenyl),
(7) —CH₂-((4-trifluoromethyl)phenyl),
(8) —CH₂-((3-trifluoromethoxy)phenyl),
(9) —CH₂-((3-trifluoromethylthio)phenyl),
(10) —CH₂-((3-trifluoromethoxy-5-thiomethyl)phenyl),
(11) —CH₂-((3-trifluoromethoxy-5-methoxy)phenyl),
(12) —CH₂-((3-trifluoromethoxy-5-methanesulfonyl)phenyl),
(13) —CH₂-((3-trifluoromethoxy-5-amino)phenyl),
(14) —CH₂-((3-trifluoromethoxy-5-aminomethanesulfonyl)phenyl),
(15) —CH₂-((3-trifluoromethoxy-5-sulfonylamino)phenyl),
(16) —CH₂-((3,5-bis-trifluoromethyl)phenyl),
(17) —CH₂-((3-fluoro-5-trifluoromethyl)phenyl),
(18) —CH(CH₃)-((3,5-bis-trifluoromethyl)phenyl),
(19) —C(CH₃)₂-((3,5-bis-trifluoromethyl)phenyl),
(20) —CH₂-(4-(2-trifluoromethyl)pyridyl),
(21) —CH₂-(5-(3-trifluoromethyl)pyridyl),
(22) —CH₂-(5-(3-trifluoromethyl)pyridazinyl),
(23) —CH₂-(4-(2-trifluoromethyl)pyridyl-N-oxide), and
(24) —CH₂-(5-(3-trifluoromethyl)pyridyl-N-oxide).

In the present invention it is preferred that R³ is hydrogen and phenyl,
where the phenyl is unsubstituted or substituted with 1-5 substituents where the substituents are independently selected from:
   (a) halo,
   (b) trifluoromethyl,
   (c) hydroxy,
   (d) C₁₋₃alkyl,
   (e) —O—C₁₋₃alkyl,
   (f) —CO₂R⁹,
   (g) —CN,
   (h) —NR⁹R¹⁰, and
   (i) —CONR⁹R¹⁰.

In the present invention it is more preferred that R³ is hydrogen and phenyl, where the phenyl is unsubstituted or substituted with 1-3 substituents where the substituents are independently selected from:
(a) halo,
(c) hydroxy,
(d) C₁₋₃alkyl,
(e) —O—C₁₋₃alkyl, and
(f) —CO₂R⁹.

In the present invention it is still more preferred that R³ is phenyl, or para-fluorophenyl.

In the present invention it is more preferred that R⁴ is selected from:
(a) hydrogen,
(b) hydroxy,
(c) —CO₂H,
(d) —CO₂C₁₋₆alkyl,
(e) —CN.

In the present invention it is more preferred that R⁵ and R⁶ are independently selected from:
(a) hydrogen,
(b) hydroxy,
(c) —CH₃,
(d) —O—CH₃, and
(e) oxo.

Especially preferred compounds of the present invention include those of the formula: 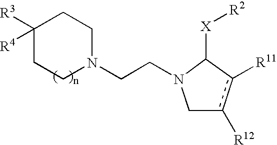
wherein the dashed line represents a single or a double bond, R¹¹ and R¹² are hydrogen or where R¹¹ and R¹² may be joined together to form a ring which is selected from:
   (a) benzene,
   (b) heterocycle
   (c) C₃₋₆cycloalkyl
and R², R³, R⁴, n and X are defined herein;
and pharmaceutically acceptable salts and individual diastereomers thereof.

Especially preferred compounds of the present invention include those of the formula: 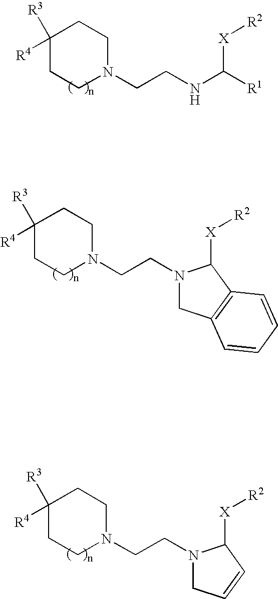
wherein R¹, R², R³, n and X are defined herein;
and pharmaceutically acceptable salts and individual diastereomers thereof.

The compounds of the instant invention have at two asymmetric centers at the amino acid part. Each such asymmetric center will independently produce two optical isomers and it is intended that all of the possible optical isomers and diastereomers in mixtures and as pure or partially purified compounds are included within the ambit of this invention. The absolute configurations of the most preferred compounds of this invention are those of the orientation as depicted: 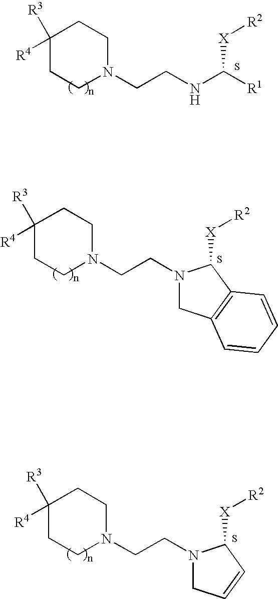
wherein the X substituent is designated as being of the “S” absolute configuration (although the designation for the X substituent may be specified as “R” if the priority for assignment of the groups at that position differs).

The independent syntheses of diastereomers and enantiomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

As appreciated by those of skill in the art, halo or halogen as used herein are intended to include chloro, fluoro, bromo and iodo. Similarly, C₁₋₈, as in C₁₋₈alkyl is defined to identify the group as having 1, 2, 3, 4, 5, 6, 7 or 8 carbons in a linear or branched arrangement, such that C₁₋₈alkyl specifically includes methyl, ethyl, n-propyl, iso-propyl, n-butyl, iso-butyl, tert-butyl, pentyl, hexyl, heptyl and octyl. Likewise, C₀, as in C₀alkyl is defined to identify the presence of a direct covalent bond. The term “heterocycle” as used herein is intended to include the following groups: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrtolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be prepared from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile are preferred. Suitable salts are found, e.g. in Remington's Pharmaceutical Sciences, 17th ed., Mack Publishing Company, Easton, Pa., 1985, p. 1418.

Exemplifying the invention is the use of the compounds disclosed in the Examples and herein.

Specific compounds within the present invention include a compound which selected from the group consisting of: the title compounds of the Examples; and pharmaceutically acceptable salts thereof and individual diastereomers thereof.

The subject compounds are useful in a method of modulating chemokine receptor activity in a patient in need of such modulation comprising the administration of an effective amount of the compound.

The present invention is directed to the use of the foregoing compounds as modulators of chemokine receptor activity. In particular, these compounds are useful as modulators of the chemokine receptors, in particular CCR-2.

The utility of the compounds in accordance with the present invention as modulators of chemokine receptor activity may be demonstrated by methodology known in the art, such as the assay for chemokine binding as disclosed by Van Riper, et al., J. Exp. Med., 177, 851-856 (1993) which may be readily adapted for measurement of CCR-2 binding.

Receptor affinity in a CCR-2 binding assay was determined by measuring inhibition of 125I-MCP-1 to the endogenous CCR-2 receptor on various cell types including monocytes, THP-1 cells, or after heterologous expression of the cloned receptor in eukaryotic cells. The cells were suspended in binding buffer (50 mM Hepes, pH 7.2, 5 mM MgCl₂, 1 mM CaCl₂, and 0.50% BSA) with and added to test compound or DMSO and ¹²⁵I-MCP-1 at room temperature for 1 h to allow binding. The cells were then collected on GFB filters, washed with 25 mM Hepes buffer containing 500 mM NaCl and cell bound ¹²⁵I-MCP-1 was quantified.

In a chemotaxis assay chemotaxis was performed using T cell depleted PBMC isolated from venous whole or leukophoresed blood and purified by Ficoll-Hypaque centrifugation followed by rosetting with neuraminidase-treated sheep erythrocytes. Once isolated, the cells were washed with HBSS containing 0.1 mg/ml BSA and suspended at 1×10⁷ cells/ml. Cells were fluorescently labeled in the dark with 2 μM Calcien-AM (Molecular Probes), for 30 min at 37° C. Labeled cells were washed twice and suspended at 5×10⁶ cells/ml in RPMI 1640 with L-glutamine (without phenol red) containing 0.1 mg/ml BSA. MCP-1 (Peprotech) at 10 ng/ml diluted in same medium or medium alone were added to the bottom wells (27 μl). Monocytes (150,000 cells) were added to the topside of the filter (30 μl) following a 15 min preincubation with DMSO or with various concentrations of test compound. An equal concentration of test compound or DMSO was added to the bottom well to prevent dilution by diffusion. Following a 60 min incubation at 37° C., 5% CO₂, the filter was removed and the topside was washed with HBSS containing 0.1 mg/ml BSA to remove cells that had not migrated into the filter. Spontaneous migration (chemokinesis) was determined in the absence of chemoattractant

In particular, the compounds of the following examples had activity in binding to the CCR-2 receptor in the aforementioned assays, generally with an IC₅₀ of less than about 1 μM. Such a result is indicative of the intrinsic activity of the compounds in use as modulators of chemokine receptor activity.

Mammalian chemokine receptors provide a target for interfering with or promoting eosinophil and/or lymphocyte function in a mammal, such as a human. Compounds which inhibit or promote chemokine receptor function, are particularly useful for modulating eosinophil and/or lymphocyte function for therapeutic purposes. Accordingly, compounds which inhibit or promote chemokine receptor function would be useful in the prevention and/or treatment of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis.

For example, an instant compound which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the compounds of the present invention. In a preferred embodiment, the disease or condition is one in which the actions of lymphocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with modulators of chemokine receptor function, include, but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum). In addition, treatment of the aforementioned inflammatory, allergic and autoimmune diseases can also be contemplated for promoters of chemokine receptor function if one contemplates the delivery of sufficient compound to cause the loss of receptor expression on cells through the induction of chemokine receptor internalization or delivery of compound in a manner that results in the misdirection of the migration of cells.

The compounds of the present invention are accordingly useful in the prevention and treatment of a wide variety of inflammatory and immunoregulatory disorders and diseases, allergic conditions, atopic conditions, as well as autoimmune pathologies. In a specific embodiment, the present invention is directed to the use of the subject compounds for the prevention or treatment of autoimmune diseases, such as rheumatoid arthritis or psoriatic arthritis.

In another aspect, the instant invention may be used to evaluate putative specific agonists or antagonists of chemokine receptors, including CCR-2. Accordingly, the present invention is directed to the use of these compounds in the preparation and execution of screening assays for compounds which modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CCR-2. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for modulating chemokine receptor activity in humans and animals comprising combining a compound of the present invention with a pharmaceutical carrier or diluent.

The present invention is further directed to the use of the present compounds in the prevention or treatment of infection by a retrovirus, in particular, herpes virus or the human immunodeficiency virus (HIV) and the treatment of, and delaying of the onset of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by, e.g., blood transfusion, organ transplant, exchange of body fluids, bites, accidental needle stick, or exposure to patient blood during surgery.

In a preferred aspect of the present invention, a subject compound may be used in a method of inhibiting the binding of a chemokine to a chemokine receptor, such as CCR-2, of a target cell, which comprises contacting the target cell with an amount of the compound which is effective at inhibiting the binding of the chemokine to the chemokine receptor.

The subject treated in the methods above is a mammal, preferably a human being, male or female, in whom modulation of chemokine receptor activity is desired. “Modulation” as used herein is intended to encompass antagonism, agonism, partial antagonism, inverse agonism and/or partial agonism. In a preferred aspect of the present invention, modulation refers to antagonism of chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

Combined therapy to modulate chemokine receptor activity and thereby prevent and treat inflammatory and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities.

For example, in the treatment or prevention of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, embrel, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamnine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the pressent invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

Examples of other active ingredients that may be combined with a compound of the present invention, either administered separately or in the same pharmaceutical compositions, include, but are not limited to: (a) VLA-4 antagonists such as those described in U.S. Pat. No. 5,510,332, WO95/15973, WO96/01644, WO96/06108, WO96/20216, WO96/22966, WO96/31206, WO96140781, WO97/03094, WO97/02289, WO 98/42656, WO98/53814, WO98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, fuirofenac, ibufenac, isoxepac, oxpinac, sulindac, tiopinac, tolmetin, zidometacin, and zomepirac), fenamic acid derivatives (flufenarnic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR-2, CCR-3, CXCR-3 and CCR-5; (j) cholesterol lowering agents such as HMG-CoA reductase inhibitors (lovastatin, simvastatin and pravastatin, fluvastatin, atorvastatin, and other statins), sequestrants (cholestyramine and colestipol), cholesterol absorption inhibitors (ezetimnibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone and pioglitazone); (l) preparations of interferon beta (interferon beta-1α, interferon beta-1β); (m) other compounds such as 5-aminosalicylic acid and prodrugs thereof, antimetabolites such as azathioprine and 6-mercaptopurine, and cytotoxic cancer chemotherapeutic agents.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally curring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require chemokine receptor modulation an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made by known procedures or as illustrated.

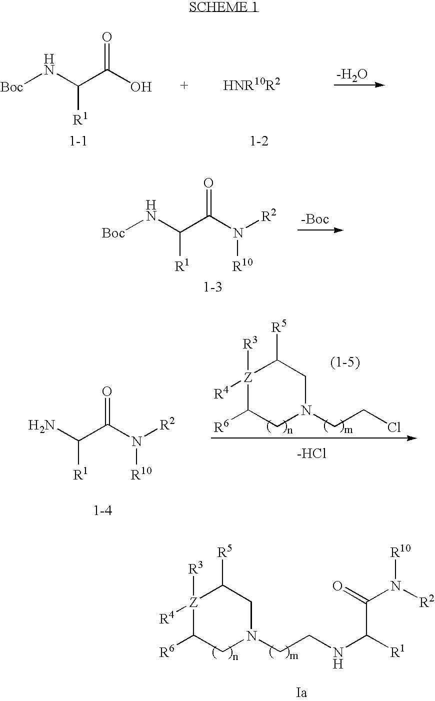

The preparation of compounds within the scope of the instant invention which bear an acyclic amino acid framework is detailed in Scheme 1. Coupling of a Boc-protected amino acid such as 1-1 with amine 1-2 to give amide 1-3 can be accomplished by the standard amide bond formation conditions using a coupling reagent such as DCC, EDC and a catalyst such as DMAP, HOBT or HOAT. De-protection of the amide 1-3 to the amine 1-4 can be carried out under standard acidic conditions, such as with TFA or 4 normal HCl dioxane solution. Alkylation of the amine 1-4 with chloroalkyl amine 1-5 under basic conditions, such as in the presence of sodium bicarbonate in hot ethanol then provides the compound of formula Ia. (Scheme 1)

Alternatively, compounds of formula Ia may be prepared by coupling of hydroxy amine 1-6 with the amine 1-2 using a coupling reagent such as as DCC, EDC and a catalyst such as DMAP, HOBT or HOAT. The resulting amide 1-7 is converted into the mesylate 1-8 by treatment of hydroxy amide with methanesulfonyl chloride. Replacement of the mesylate 1-8 with the diamine 1-9 then provides the compound of formula Ia (Scheme 1a)

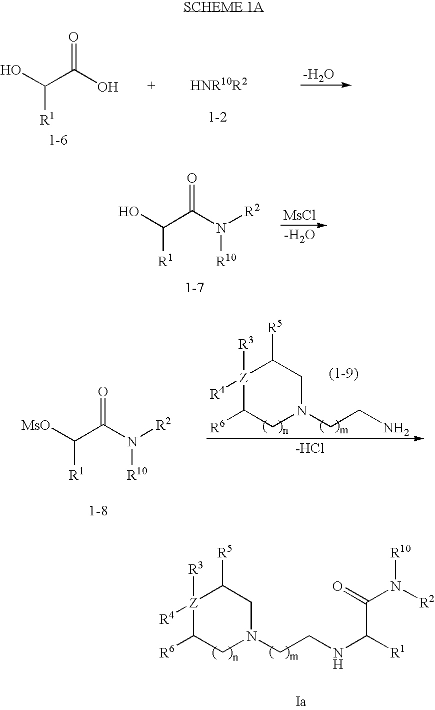

For the preparation of the 1-6 not commercially available, a convenient route is developed starting from treatment of ethyl glyoxalate solution 1-10 with zinc reagent 1-11 to give the hydroxy ester 1-12, as depicted in Scheme 1B. Saponification of the ester 1-12 then provides the acid 1-6 which can be then converted to the compound of formula Ia according to Scheme 1A.

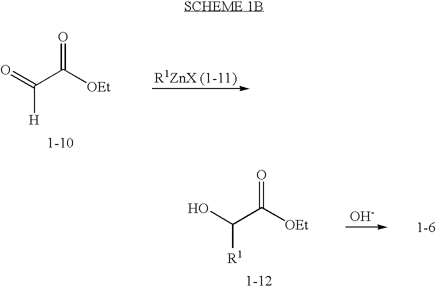

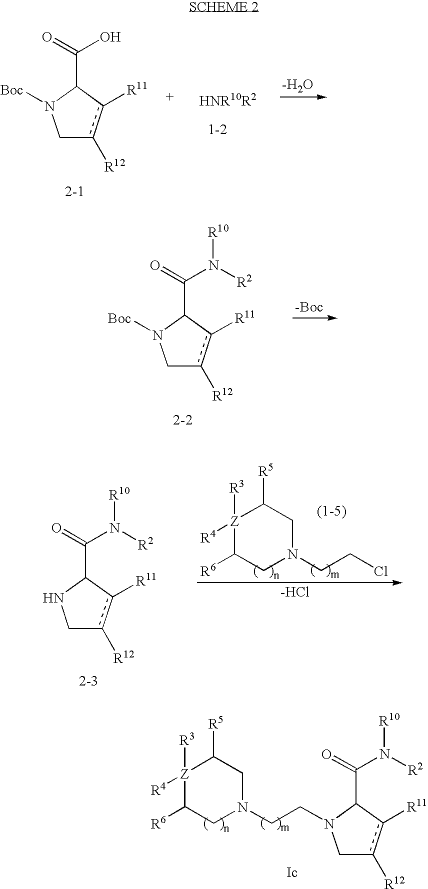

As depicted in Scheme 2, the Boc-protected cyclic amino acid 2-1 can be converted to the amide 2-2 by the standard amide bond formation conditions using a coupling reagent such as DCC, EDC and a catalyst such as DMAP, HOBT or HOAT. De-protection of the amide 2-2 to the amine 2-3 can be carried out under standard acidic conditions, such as with TFA or 4 normal HCl dioxane solution. The amine 2-3 could be subjected to previously mentioned condition with chloroalkyl amine 1-5 to form the compound of the formula Ic.

The compounds of formula Ic can also be formed by an alternative route, as depicted in Scheme 2A. The intermediate amine 2-3 is alkylated with chloroaldehyde 2-4 to form the chloroalkyl amine 2-5 under a variety of conditions, including sodium triacetoxyborohydride or sodium cyanoborohydride. Replacement of the chloride 2-5 with the amine 2-6 in the presence of sodium bicarbonate in hot ethanol then provides the compound of the formula Ic.

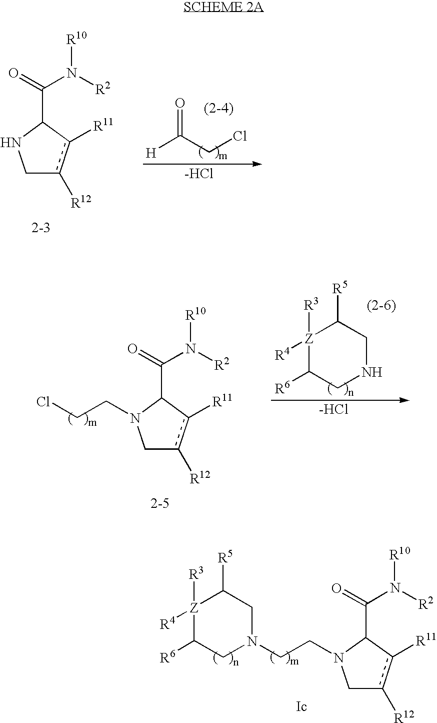

The above procedure is also used to make the compound of the formula IIa, as depicted in Scheme 3. A variety of the amine 3-1 such as primary, secondary and tertiary amine can be used to react with the chloride 2-5.

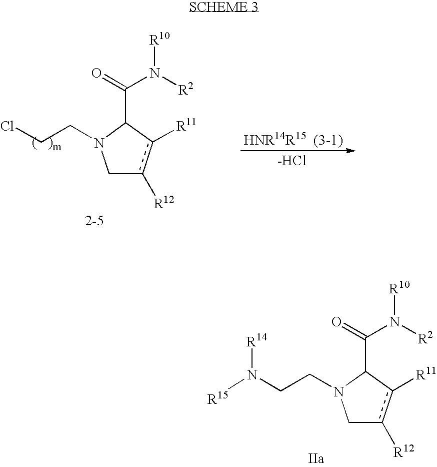

In some cases the order of carrying out the foregoing reaction schemes may be varied to facilitate reaction or to avoid unwanted reaction products. The following examples are provided for the purpose of further illustration only and are not intended to be limitations on the disclosed invention.

Concentration of solutions was generally carried out on a rotary evaporator under reduced pressure. Flash chromatography was carried out on silica gel (230-400 mesh). NMR spectra were obtained in CDCl₃ solution unless otherwise noted. Coupling constants (J) are in hertz (Hz). Abbreviations: diethyl ether (ether), triethylamine (TEA), N,N-diisopropylethylamine (DIEA) saturated aqueous (sat'd), room temperature (rt), hour(s) (h), minute(s) (min).

The following are representative Procedures for the preparation of the compounds used in the following Examples or which can be substituted for the compounds used in the following Examples which may not be commercially available.

### EXAMPLE 1

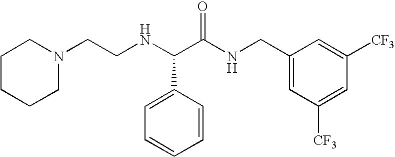

### (2S)-N-[3,5-bis(trifluoromethyl)benzyl]-2-phenyl-2-[(2-pipeddin-1 -ylethyl)amino]ethanamide

A mixture of 251 mg (1.0 mmol) (S)-N-Boc-phenylglycine, 280 mg (1.0 mmol) 3,5-bis(trifluoromethyl)benzylamine hydrochloride, 143 mg (1.1 mmol) di-isopropylethylamine, 283 mg (1.5 mmol) 1-[3-(dimethylamino)propyl]3-ethylcarbodiimide hydrochloride in 20 mL of methylene chloride was stirred for 4 hrs. The mixture was diluted with 50 mL of methylene chloride. The organic phase was washed with water, 3N aq. HCl (2×50 mL), sat. NaHCO3 (100 mL) and brine (100 mL), dried over Na2SO4, evaporated to afford the coupling product as white solid which was dissolved in 20 mL of 4N HCl in dioxane. The mixture was stirred at RT for 2 h and evaporated to afford a white solid. The entire material was dissolved a vial containing 500 mg of sodium bicarbonate, 100 mg of N-chloroethylpiperidine hydrochloride and 5 mL of ethanol/water (95/5). The mixture was heated at 60 C overnight, cooled at RT, filtered, washed with ethanol. The filtrates were combined and evaporated. The residue was purified on preparative TLC (1000 Micron Silica Gel; developed by 10% [aq.ammonia/methanol 1/9] in methylene chloride). 45 mg of the title product was obtained as light brown oil which was further converted into hydrochloride salt for delivery purpose. 1H-NMR of the title compound (free base in CDCl3): 1.48-1.51 (m, 6H), 2.32-2.42 (m, 8H), 2.60-2.80 (m, 2H), 4.25 (s, 1H), 4.40-4.50 (dd, 2H), 7.20-7.40 (m, 5H), 7.64 (s, 2H), 7.75 (s, 1H), 8.18 (wide, 1H).

Mass Spectrum (NH₃-CI): m/z 488.2 (M+1).

The same procedure was followed in the preparation of the following examples.

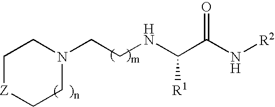

| | | | | | MS |
|---|---|---|---|---|---|
| | | | | | M⁺ + |
| Ex. | R¹ | R² | n m | Z | 1 |
| 2 | 4-F-Ph | 3,5-Bis-CF₃-Benzyl | 1 1 | CH₂ | 506.2 |
| 3 | 4-F-Ph | 3,5-Bis-CF₃-Benzyl | 0 1 | CH₂ | 492.2 |
| 4 | 4-F-Ph | 3,5-Bis-CF₃-Benzyl | 1 2 | CH₂ | 520.2 |
| 5 | 4-HO-Ph | 3,5-Bis-CF₃-Benzyl | 1 1 | CH₂ | 504.2 |
| 6 | 3-Thienyl | 3,5-Bis-CF₃-Benzyl | 1 1 | CH₂ | 494.2 |
| 7 | 2-Thienyl | 3,5-Bis-CF₃-Benzyl | 1 1 | CH₂ | 494.2 |
| 8 | Ph | 3,5-Bis-CF₃—C₆H₄CH | 1 1 | CH₂ | 502.2 |
| | | (Me) | | | |
| 9 | 4-Cl-Ph | 3,5-Bis-CF₃-Benzyl | 1 1 | CH₂ | 522.2 |
| 10 | Cyclo-hexyl | 3,5-Bis-CF₃-Benzyl | 1 1 | CH₂ | 494.3 |
| 11 | Me | 3,5-Bis-CF₃-Benzyl | 1 1 | CH₂ | 426.2 |
| 12 | i-Propyl | 3,5-Bis-CF₃-Benzyl | 1 1 | CH₂ | 452.2 |
| 13 | BocNH(CH₂)₄ | 3,5-Bis-CF₃-Benzyl | 1 1 | CH₂ | 582.3 |
| 14 | CbzNH(CH₂)₃ | 3,5-Bis-CF₃-Benzyl | 1 1 | CH₂ | 602.3 |
| 15 | H₂NCONH2— | 3,5-Bis-CF₃-Benzyl | 1 1 | CH₂ | 512.2 |
| | (CH₂)₃— | | | | |
| 16 | 3-Thienyl | 3,5-Bis-CF₃—C₆H₄CH(Me) | 1 1 | CH₂ | 508.2 |
| 17 | Ph | 3,5-Bis-CF₃-Benzyl | 1 1 | O | 490.2 |
| 18 | Ph | 3,5-Bis-CF₃-Benzyl | 2 1 | CH₂ | 474.4 |
| 19 | PhCH₂ | 3,5-Bis-CF₃-Benzyl | 1 1 | CH₂ | 520.2 |
| 20 | Ph | 3-CF₃-Benzyl | 1 1 | CH₂ | 419.2 |

### EXAMPLE 21

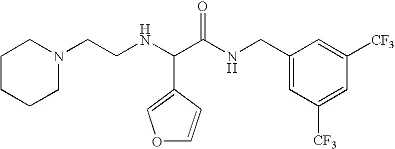

### N-[3,5-bis(trifluoromethyl)benzyl]-2-(3-furyl)-2-[(2-piperidin-1-ylethyl)amino]acetamide

Step 1: Ethyl α-hydroxy-3-Furylacetate

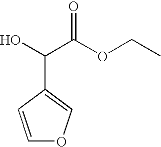

To a stirred solution of n-butyllithium (1.6 M, 34 mL, 55 mmol) in TVF at −78 C was added a neat solution of 3-bromofuran (7.35 g, 50 mmol) in 10 minutes. The mixture was stirred at −78 C for 0.5 h. A solution of Zinc chloride (1.0 M, 50 mL) in ether was added and continued to stirr for 0.5 h. Ethyl gloxalate (50%, 12 g, 60 mmol) in toluene was added. The reaction was stirred for 10 min. at −78 C, warmed to room temperature for 30 min. and quenched with sat. aq. NH4Cl, extracted with ether, dried over Na2SO4 and evaporated. MPLC (40% EtOAc/Hexane) gave the title compound as colorless oil (2.20 g). ¹H NMR (300 MHz, CDCl3): δ1.25 (t, 3H), 3.20 (w, 1H), 4.23 (q, 2H), 5.14 (d, 1H), 6.41 (s, 1H), 7.38 (s, 1H), 7.47 (s, 1H).
Step 2:

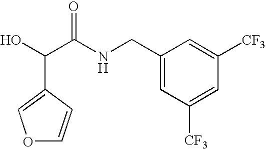

1.70 g (10 mmol) of the above ester (from Example IV, step 1) was heated at 80 C for 2 h. in a mixture of lithium hydroxide monohydrate (0.80 g, 20 mmol) and 20 mL of dioxane/water (1/1), cooled to RT and acidified with 2N aq. Hcl, evaporated to remove dioxane. The residue was extracted with ethyl acetate and washed with 2N aq. Hcl and brine, dried over Na2SO4 and evaporated. The residue was mixed with 2.80 g (10 mmol) of 3,5-bis(trifluoromethyl)-benzylamine hydrochloride, 2.0 g (20 mmol) of N-methyl morpholine, 2.30 g (12 mmoL) of EDAC in 20 mL of methylene chloride. The mixture was stirred at RT for 2 h and diluted with 100 mL of DCM. The organic phase was washed with water, 2N aq. Hcl and brine, dried over Na2SO4 and evaporated. MPLC (50% EtOAc/Hexane) gave the title compound as a white solid (1.20 g). ¹H NMR (300 M, CDCl₃): δ4.59 (d, 2H), 5.16 (s, 1H), 6.40 (s, 1H), 7.00 (wide, 1H), 7.41 (s, 1H), 7.51 (s, 1H), 7.66 (s, 2H), 7.78 (s, 1H).
Step 3:

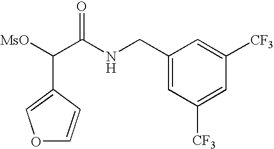

To a cool (0 C) solution of the above alcohol (367 mg, 1.0 mmol) (Example IV, Step 2), triethylamine (300 mg, 3.0 mmol) and DMAP (2.0 mg) in 10 mL of methylene chloride was added a solution of mesyl chloride (125 mg, 1.1 mmol) in 2 mL of methylene chloride. The mixture was stirred at 0 C for 1 h, washed with 2N aq. Hcl and water, dried over Na2SO4 and evaporated to afford a yellow solid. ¹H-NMR (300 MHz, CDCl₃): δ2.99 (s, 3H), 4.50 (m, 2H), 6.00 (s, 1H), 6.22 (s, 1H), 7.48 (s, 1H), 7.65 (s, 1H), 7.72 (s, 2H), 7.78 (s, 1H).
Step 4:

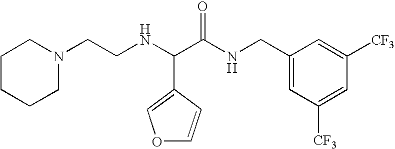

The above crude mesylate (200 mg) (Example IV, Step 3) was stirred with 1-(2-aminoethyl)piperidine (1.0 g) in 5 mL of methylene chloride for 1 h, evaporated, purified by preparative LC (10% [aq. ammonia/MeOH 1/9]/DCM). 57 mg of the title compound was obtained as a yellow oil. ¹H NMR (300 MHz, CDCl₃): δ1.38-1.52 (m, 6H), 2.31-2.44 (m, 6H), 2.65-2.74 (m, 2H), 4.20 (s, 1H), 4.49-4.62 (dd, 2H), 6.37 (s, 1H), 7.36 (s, 1H), 7.40 (s, 1H), 7.65 (s, 2H), 7.73 (s, 1H), 8.19 (wide, 1H). Mass Spectrum (NH₃-CI): m/z 478.2 (M+1).

### EXAMPLE 22

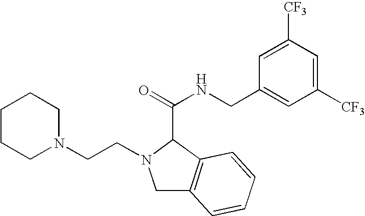

### (2S)-N-[3,5-bis(trifluoromethyl)benzyl]-1-[2-(4-hydroxypiperidin-1-yl)ethyl]-isoquinoline-2-carboxamide

Step 1: Coupling

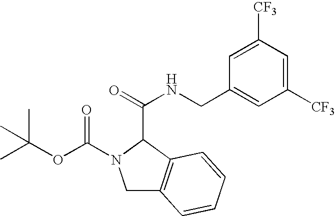

A mixture of 5.0 g (19 mmol) (R,S)-N-Boc-1,3-dihydro-2H-isoindole carboxylic acid, 5.57 g (20 mmol) 3,5-bis(trifluoromethyl)benzylamine hydrochloride, 3.64 mL (20 mmol) di-iso-propyl ethyl amine, 5.46 g (29 mmol) 1-[3-(dimethylamino)propyl]3-ethylcarbodiimide hydrochloride in 120 mL of methylene chloride was stirred for 4 hours. The mixture was diluted with 200 mL of methylene chloride. The organic phase was washed with water, 3N aq. HCl (2×300 mL), sat. NaHCO3 (300 mL) and brine (300 mL), dried over Na2SO4, evaporated to afford the coupling product as white solid This crude material was brought to next reaction without further purification. Mass Spectrum (NH₃-CD): m/z 489.2 (M+1).
Step 2: De-protection

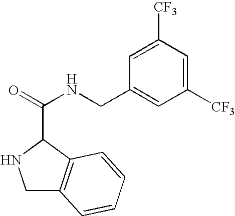

The above product (from Example 22, step 1) was dissolved in 100 mL of 4N HCl in dioxane. The mixture was stirred at RT for 2 h, evaporated to afford 8.61 g white solid. 2.0 g of the solid was saved for other purpose, the rest was was suspended in methylene chloride and treated with sat. aq. NaHCO3. The organic phase was dried in vacuum and 4.85 g free amino amide was obtained. Mass Spectrum (NH₃-CI): m/z 339.2 (M+1).
Step 3: Reductive Amination

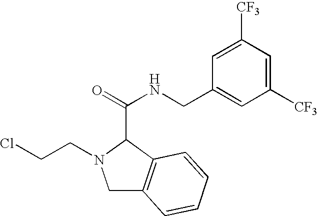

In a flask was loaded 4.85 g (12.5 mmol) of the above entire amine (from Example 22, step 2), 3.9 g (25 mmol) of chloroacetaldehyde (50% solution in water), 25 g of molecular sieves (4A) and 100 mL of methylene chloride. The resulting was stirred for 10 min, 5.3 g (25 mmol) of sodium triacetoxyborohydride was then added in one portion. After stirred for 1 h, the mixture was quenched with water. The mixture was filtered, the solid was washed with methylene chloride and then discarded. The filtrates were separated and organic phase was washed with sat. NaHCO3, water and brine, dried over Na2SO4, evaporated to dryness. Flash chromatography (10% MeOH/DCM) afforded 3.14 g (56%) of the title compound as a white solid. ¹H NMR (300 MHz, CDCl₃): δ3.14-3.18 (m, 21), 3.69-3.73 (m, 2H), 3.80-3.86 (m, 1H), 4.50-4.60 (m, 2H), 7.22-7.32 (m, 3H), 7.50-7.52 (d, 1), 7.63 (s, 2H), 7.73 (s, 1H), 8.19 (wide, 1H). Mass Spectrum (NH₃-CI): m/z 451.2 (M+1).
Step 4: Alkylation

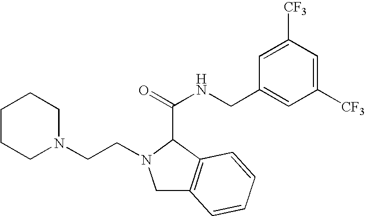

100 mg (0.222 mmol) of the above chloroethylamine (Example 22, step 3), 85 mg (1.0 mmol) of piperidine and 100 mg of sodium bicarbonate in 5 mL of ethanol/water (95/5) was stirred at 80° C. for 5 h, filtered and evaporated to dryness. The residue was purified on preparative TLC (10%[aq.NH4OH/MeOH 1/9]/DCM), 42 mg of the title compound was obtained as free diamine. ¹H NMR (300 MHz, CDCl₃): δ1.40-1.46 (m, 5H), 2.40-2.60 (m, 5H), 2.80-2.90 (m, 1H), 2.95-3.10 (m, 1H), 3.80-3.90 (m, 1H), 4.40-4.70 (m, 4H), 7.20-7.30 (m, 3H), 7.50 (d, 1H), 7.59 (s, 2H), 7.71(s, 1H), 8.64 (wide, 1H). Mass Spectrum (NH₃-CI): m/z 500.2 (M+1).

Similar analogs were made as the same way as example 22 starting from common intermediate (Example 22, step 3) and the corresponding amines.

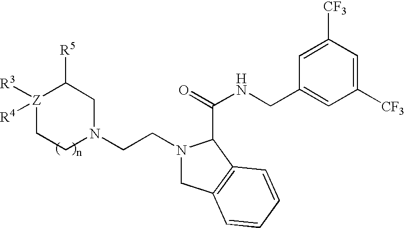

| | | | | | | MS |
|---|---|---|---|---|---|---|
| Ex. | R³ | R⁴ | R⁵ | n | Z | M⁺ + 1 |
| 23 | H | H | H | 0 | C | 486.2 |
| 24 | H | H | OH | 1 | C | 516.2 |
| 25 | Ph | H | H | 1 | C | 576.2 |
| 26 | H | H | Ph | 0 | C | 562.2 |
| 27 | H | OH | H | 1 | C | 516.2 |
| 28 | CO₂Me | H | H | 1 | C | 558.2 |
| 29 | CO₂Me | | H | 1 | N | 559.2 |
| 30 | Ph | | H | 1 | N | 577.2 |
| 31 | 4-F-Ph | | H | 1 | N | 595.2 |
| 32 | | | H | 1 | O | 502.2 |
| 33 | H | H | H | 1 | C | 514.2 |

### EXAMPLE 34

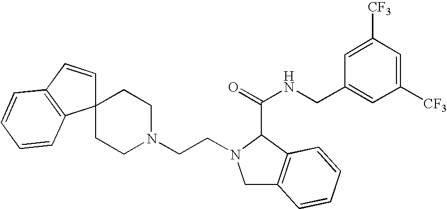

100 mg (0.222 mmol) of the chloroethylamine (Example 22, step 3), 100 mg (0.45 mmol) of spiroindenepiperidine (available from Arch Corp) hydrochloride and ˜100 mg of sodium bicarbonate in 5 mL of ethanol/water (95/5) was stirred at 80° C. for 5 h, filtered and evaporated to dryness. The residue was purified on preparative TLC (10%[aq.NH4OH/MeOH 1/9]/DCM), 82 mg of the title compound was obtained as free diamine. Mass Spectrum (NH₃-CI): m/z 600.2 (M+1).

The similar procedure was also used to prepare the compounds of the following formula by treatment of the chloroethyl amine (Example 22, step 3) with a variety of acyclic, primary and secondary amines in the presence of sodium bicarbonate in hot (80° C.) ethanol for 5 hours. The isolated yield ranges from 30% to 60% upon the structure of the amine and also varies from batch to batch.

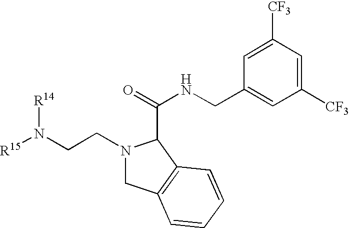

| Ex. | R¹⁴ | R¹⁵ | MS: M⁺ + 1 |
|---|---|---|---|
| 35 | Me | Me | 460.2 |
| 36 | PhCH₂ | H | 522.2 |
| 37 | (4-Pyridyl)CH₂CH₂ | H | 537.2 |
| 38 | 4-HOC₆H₄CH₂CH₂ | H | 552.2 |
| 39 | 4-H₂NC₆H₄CH₂CH₂ | H | 551.3 |
| 40 | PhCH₂CH₂ | H | 536.2 |
| 41 | PhCH₂CH₂ | Me | 550.2 |
| 42 | Ph₂CHCH₂CH₂ | H | 626.3 |
| 43 | PhCH₂CH₂CH₂ | Me | 564.2 |
| 44 | PhCH₂CH₂CH₂CH₂ | H | 564.2 |
| 45 | PhCH₂CH₂CH(CH₃) | H | 564.2 |
| 46 | 4-ImidazoleCH₂CH₂ | H | 526.2 |
| 47 | cyclohexyl | H | 514.2 |
| 48 | cyclohexyl | Me | 528.2 |
| 49 | cyclohexylCH₂ | H | 528.2 |
| 50 | Iso-propyl | Me | 488.2 |

### EXAMPLE 51

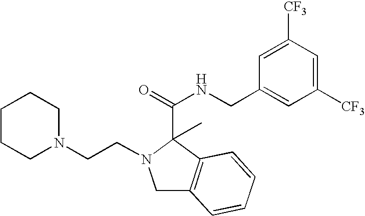 Step 1:

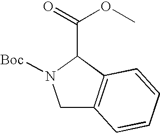

A mixture of (R,S)-N-Boc-1,3-dihydro-2H-isoindole carboxylic acid (1.6 g, 6.0 mmol), iodomethane (1.0 g, 7.0 mmol), potassium carbonate (5.0 g) in 20 mL of DMF was stirred overnight. 200 mL of water and 200 mL of ethyl acetate were added. The organic phase was washed with water (3 times) and brine, dried over Na2SO4, evaporated to afford a white solid (1.20 g). ¹H NMR (300 MHz, CDCl₃): δ1.50 (d, 9H), 3.74 (d, 3H), 4.76 (m, 2H), 5.45 (d, 1H), 7.20-7.40 (s, 4H).
Step 2:

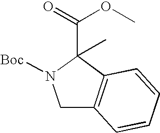

To a stirred solution of the above ester (1.1 g, 4.0 mmol) (Example LXI, Step 1) in 20 mL of THF at −78 C was added dropwise a solution of sodium bis(trimethylsilyl)amide (1.0 M, 5.0 mL, 5 mmol) in THF. The mixture was stirred at −78 C for 10 min. 1.0 g (7.0 mmol) of iodomethane was added sropwise. The resulting solution was stirred at −78 C for 1 h. and then warmed to RT, evaporated. Ethyl acetate/water work-up gave a yellow residue which was purified on MPLC (20% Ethyl Acetate/Hexane). 0.60 g of the title compound was obtained as a yellow oil. ¹H NMR (300 MHz, CDCl₃): δ1.47, 1.51 (ss, 9H), 1.83,1.90 (ss, 3H), 3.64,3.65 (ss, 3H), 4.60-4.83 (m, 2H), 7.21-7.31 (m, 4H).
Step 3:

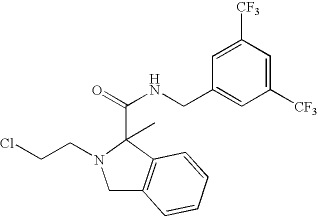

The above ester (0.60 g, 2.0 mmol) (Example IX, Step 2) was heated with lithium hydroxide monohydrate (160 mg, 4 mmol) in a mixture of dioxaneiwater (3/1, 20 mL) overnight. The solution was acidified with 2N aq. Hcl to PH>3 and extracted with ethyl acetate (3×). The organic phases were combined and washed with water and brine, dried over Na2SO4, evaporated. The residue was dissolved in 20 mL of methylene chloride, bis(trifluoromethyl)benzylamine hydrochloride (600 mg, 2.1 mmol) and EDAC (570 mg, 3.0 mmol) was added. The resulting mixture was stirred for 2 h, quenched with water, diluted with methylene chloride, separated. The organic phase was washed with 2N aq. Hcl, water and brine, dried over Na2SO4, evaporated. The residue was treated with 4N Hcl in dioxane (20 mL) for 1 h, evaporated, dried in vacuum. The residue was stirred with 0.38 mL (3 mmol) of chloroacetaldehyde (50% solution in water), 5.0 g of molecular sieves (4A) in 20 mL of methylene chloride for 30 min, 0.45 g (4 mmol) of sodium triacetoxyboride was added The mixture was stirred for 30 min, filtered through a pad of celite, washed with methylene chloride. The filtrates were combined and washed with sat. NaHCO3, water and brine, dried over Na2SO4, evaporated, dried in vaccum. 0.3 g of the title compound was obtained as a white solid. ¹H- NMR (300 MHz, CDCl₃): δ1.49 (s, 3H), 3.05-3.07 (m, 2H1), 3.75-3.79 (m, 4H), 4.44-4.58 (m, 4H), 7.25-7.29 (m, 3H), 7.40-7.41 (m, 1H), 7.58 (s, 2H), 7.70 (s, 1H), 8.50 (wide, 1H). Mass Spectrum (NH₃-CI): m/z 464.2 (M+1).
Step 4:

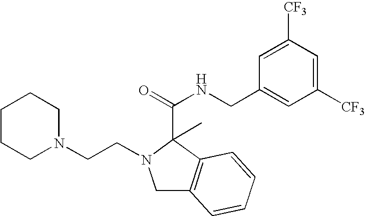

60 mg of the above chloroethylaminoamide (Example LXI, step 3) was heated in 0.3 mL of piperidine at 80° C. for 3 h, evaporated to remove excess of piperidine. The residue was purified on preparative TLC (10%[aq.NH4OH/MeOH 1/9]/DCM), 40 mg of the title compound was obtained as a yellow oil. ¹H NMR (300 MHz, CDCl₃): δ1.41-1.48 (m, 8H), 2.38-2.46 (m, 6H), 3.76,3.80 (ss, 1H), 4.40-4.48 (m, 2H), 4.64-4.66 (m, 1H), 7.21-7.26 (m, 3H), 7 (m, 3H),7.50 (d, 1H),7.59 (s,2H), 7.71(s, 1H), 8.64 (wide, 1H). Mass Spectrum (NH₃-CI): m/z 500.2 (M+1).

### EXAMPLE 52

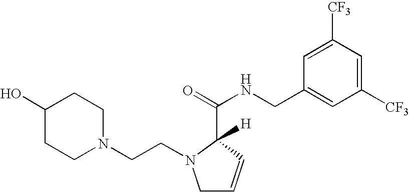

### (2S)-N-[3,5-bis(trifluoromethyl)benzyl]-1-[2-(4-hydroxypiperidin-1-yl)ethyl]-2,5-dihydro-1H-pyrrole-2-carboxamide

Step 1: Coupling

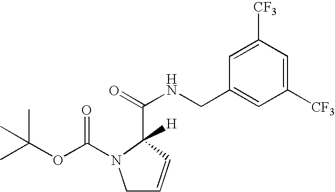

A mixture of 5.33 g (25 mmol) N-Boc-3,4-dedydroproline , 7.30 g (26 mmol) 3,5-bis(trifluoromethyl)benzylamine hydrochloride, 3.54 g (26 mmol) HOAt, 5.3 mL (30 mmol) di-isopropylethylamine, 5.73 g (30 mmol) 1-[3-(dimethylamino)propyl]3-ethylcarbodiimide hydrochloride in 200 mL of methylene chloride was stirred for 2 hours. The mixture was diluted with 200 mL of methylene chloride. The organic phase was washed with 2N aq. HCl (2×300 mL), water (2×300 mL) and brine (2×300 mL), dried over Na2SO4, evaporated to afford the coupling product as white solid.
Step 2: De-protection

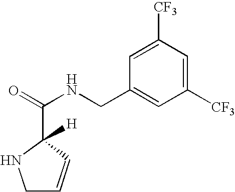

The above product (from Example 52, step 1) was dissolved in 100 mL of 4N HCl in dioxane. The mixture was stirred at RT over 1 h, evaporated to leave a white solid which was suspended in methylene chloride and treated with sat. aq. NaHCO3. Organic phase was dried in vacuum and used directed for the following reactions. Mass Spectrum (NH₃-CI): m/z 339.2 (M+1).
Step 3: Reductive Amination

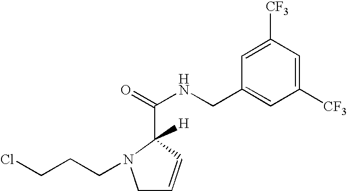

In a flask was loaded the above entire aminoamide (from Example 52, step 2), 3.8 mL (30 mmol) of chloroacetaldehyde (50% solution in water), 25 g of molecular sieves (4A) and 100 mL of methylene chloride. The resulting was stirred for 30 min, 9.7 g (45 mmol) of sodium triacetoxyboride was then added in one portion. After stirred for 1 h, the mixture was quenched with water. The mixture was filtered, the solid was washed with methylene chloride and then discarded. The filtrates were separated and organic phase was washed with sat. NaHCO3, water and brine, dried over Na2SO4, evaparated to afford a solid. ¹H NMR (300 MHz, CDCl₃): δ3.84-3.88 (m, 3H), 4.63-4.68 (m, 5H), 6.15-6.17 (m, 1H), 6.45 (wide, 1H), 6.73-6.69 (t, 1H), 6.68-6.69 (d, 1H), 6.91-6.93 (m, 1H), 7.78 (s, 3H). Mass Spectrum (NH₃-CI): m/z 401.2 (M+1).
Step 4:Alkylation

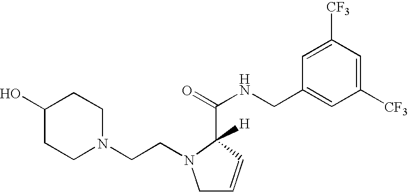

150 mg of the above chloroethylaminoamide, 400 mg of 4-hydroxypiperidine hydrochloride and 800 mg of sodium bicarbonate in 5 mL of ethanol/water (95/5) was stirred at: 80° C. for 5 h, filtered and evaporated. The residue was purified on preparative TLC (10%[aq.NH4OH/MeOH 1/9]/DCM), 72 mg of the desired product was obtained as an gummy solid. It was converted into hydrochloride salt with 4N HCl/dioxane and delivered for bioassays. Mass Spectrum (NH₃-CI): m/z 466.2 (M+1).

Similar analogs were made as the same way as Example 52 starting from common intermediate (Example 52, step 3).

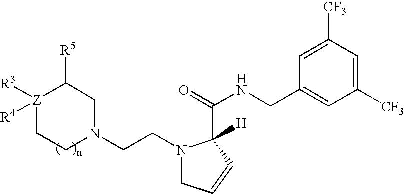

| | | | | | | MS: |
|---|---|---|---|---|---|---|
| Ex. | R³ | R⁴ | R⁵ | n | Z | M⁺ + 1 |
| 53 | H | H | H | 0 | C | 436.1 |
| 54 | H | H | Ph | 0 | C | 512.2 |
| 55 | H | H | PhCH₂ | 1 | C | 540.2 |
| 56 | H | H | OH | 1 | C | 466.2 |
| 57 | H | H | NHBoc | 0 | C | 551.2 |
| 58 | H | H | OH | 0 | C | 452.2 |
| 59 | H | H | o-MePh | 0 | C | 526.2 |
| 60 | HOCH₂ | H | Ph | 0 | C | 542.2 |
| 61 | PhCH₂CH₂CH₂ | OH | H | 1 | C | 584.3 |
| 62 | H | H | Ph | 1 | C | 463.2 |
| 63 | Ph | H | H | 1 | C | 526.2 |
| 64 | H | H | Ph | 1 | C | 526.2 |
| 65 | NHBoc | H | H | 1 | C | 565.3 |
| 66 | CO₂Me | H | H | 1 | C | 508.2 |
| 67 | H | H | CO₂Me | 1 | C | 508.2 |
| 68 | CO₂Me | | H | 1 | N | 509.2 |
| 69 | Ph | | H | 1 | N | 523.2 |
| 70 | | | H | 1 | O | 452.2 |
| 71 | H | H | H | 2 | C | 464.2 |

The compounds of the following formula were prepared according to the same procedure as those of the preparation of the example 52 from the corresponding amines.

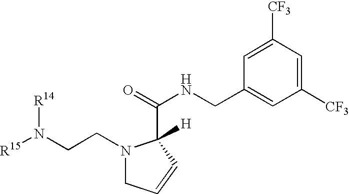

| Ex. | R¹⁴; R¹⁵ | M⁺ + 1 |
|---|---|---|
| 72 | Me; Me | 410.1 |
| 73 | H; PhCH₂CH₂ | 486.2 |
| 74 | Me; PhCH₂CH₂ | 500.2 |
| 75 | 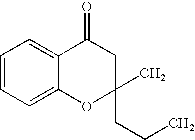 | 582.2 |
| 76 | 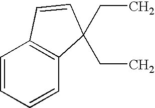 | 550.2 |
| 77 | 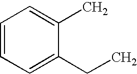 | 498.2 |

### EXAMPLE 78

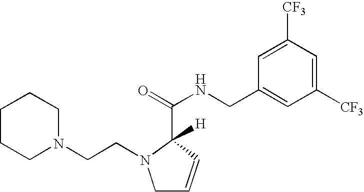

### N-[3,5-bis(trifluoromethyl)benzyl]-1-(2-piperidin-1-ylethyl)-L-prolinamide

A mixture of 215 mg (1.0 mmol) (S)-N-Boc-proline, 280 mg (1.0 mmol) 3,5-bis(trifluoromethyl)benzylamine hydrochloride, 143 mg (1.1 mmol) di-isopropylethylamine, 283 mg (1.5 mmol) 1-[3-(dimethylamino)propyl]3-ethylcarbodiimide hydrochloride in 20 mL of methylene chloride was stirred for 4 hrs. The mixture was diluted with 50 mL of methylene chloride. The organic phase was washed with water, 3N aq. HCl (2×50 mL), sat. NaHCO3 (100 mL) and brine (100 mL), dried over Na2SO4, evaporated to afford the coupling product as white solid which was dissolved in 20 mL of 4N HCl in dioxane. The mixture was stirred at RT for 2 h and evaporated to afford a white solid. The entire material was dissolved a vial containing 500 mg of sodium bicarbonate, 100 mg of N-chloroethylpiperidine hydrochloride and 5 mL of ethanol/water (95/5). The mixture was heated at 60 C overnight, cooled at RT, filtered, washed with ethanol. The filtrates were combined and evaporated. The residue was purified on preparative TLC (1000 Micron Silica Gel; developed by 10% [aq. ammonia/methanol 1/9] in methylene chloride). 27 mg of the title product was obtained as light brown oil which was further converted into hydrochloride salt for delivery purpose. ¹H-NMR of the title compound (free base in CDCl₃): 1.36-1.40 (m, 6H), 1.60-1.80 (m, 2H), 1.90-2.05 (m, 1H), 2.06-2.50 (m, 8H), 2.60-2.80 (m, 2H), 3.18-3.22 (m, 2H), 4.38-4.72 (dd, 21), 7.70 (s, 2H), 7.73 (s, 1H), 8.95 (wide, 1H). Mass Spectrum (NH₃-CI): m/z 452.2 (M+1).

### EXAMPLE 79

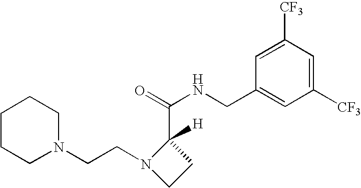

### (2S)-N-[3,5-bis(trifluoromethyl)benzyl]-1-(2-piperidin-1-ylethyl)azetidine-2-carboxamide

The title compound was prepared using the same procedure as detailed in Example 78 with the replacement of Boc-L-proline by Boc-L-azetidine carboxylic acid. LC-MS for C₂₀H₂₅F₆N₃O. [M+H+] calculated 438.2, found 438.2.

### EXAMPLE 80

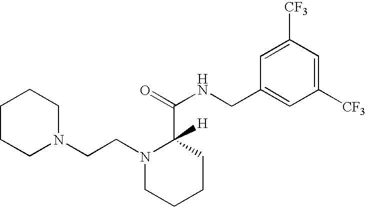

### (2S)-N-[3,5-bis(trifluoromethyl)benzyl]-1-(2-piperidin-1-ylethyl)piperidine-2-carboxamide

The title compound was prepared using the same procedure as detailed in Example 78 with the replacement of Boc-L-proline by Boc-L-piperidine carboxylic acid. LC-MS for C₂₂H₂₉F₆N₃O. [M+H+] calculated 466.2, found 466.2.

### EXAMPLE 81

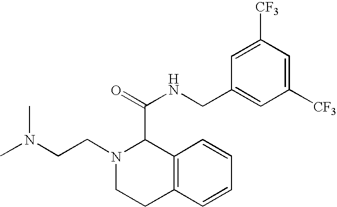

### N-[3,5-bis(trifluoromethyl)benzyl]-2-[2-(dimethylamino)ethyl]-1,2,3,4-tetrahydroisoquinoline-1-carboxamide

The title compound was prepared using the same procedure as detailed in Example 78 with the replacement of Boc-L-proline and N-chloroethylpiperidine by Boc-1,2,3,4-tetrahydroisoquinoline-1-carboxylic acid and N-chloroethyl dimethylamine. LC-MS for C₂₃H₂₅F₆N₃O. [M+H+] calculated 474.2, found 474.2.

### EXAMPLE 82

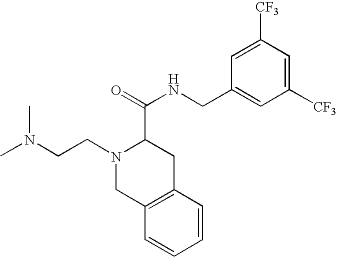

### N-[3,5-bis(trifluoromethyl)benzyl]-2-[2-(dimethylamino)ethyl]-1,2,3,4-tetrahydroisoquinoline-3-carboxamide

The title compound was prepared using the same procedure as detailed in Example 78 with the replacement of Boc-L-proline and N-chloroethylpiperidine by Boc-1,2,3,4-tetrahydroisoquinoline-3-carboxylic acid and N-chloroethyl dimethylamine. LC-MS for C₂₃H₂₅F₆N₃O. [M+H+] calculated 474.2, found 474.2.

### EXAMPLE 83

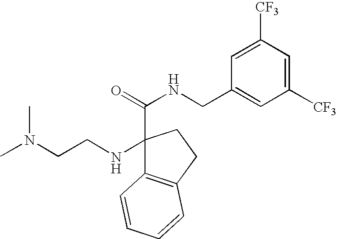

### N-[3,5-bis(trifluoromethyl)benzyl]-1-{[2-(dimethylamino)ethyl]amino}indane-1-carboxamide

The title compound was prepared using the same procedure as detailed in Example 78 with the replacement of Boc-L-proline and N-chloroethylpiperidine by Boc-indane-1-carboxylic acid and N-chloroethyl dimethylamine. LC-MS for C₂₃H₂₅F₆N₃O. [M+H+] calculated 474.2, found 474.2.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound of the formula I: 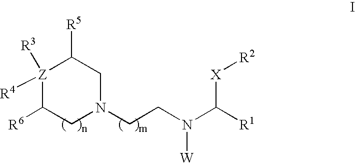
wherein:
X is selected from the group consisting of:
—NR¹⁰—, —O—, —CH₂O—, —CONR¹⁰—, —NR¹⁰CO—, —CO₂—, —OCO—, —CH₂(NR¹⁰)CO—, —N(COR¹⁰)—, —CH₂N(COR¹⁰)—, phenyl, and C₃₋₆ cycloalkyl,
where R¹⁰ is independently selected from: hydrogen, C₁₋₆ alkyl, benzyl, phenyl, and C₁₋₆ alkyl-C₃₋₆ cycloalkyl,
which is unsubstituted or substituted with 1-3 substituents where the substituents are independently selected from: halo, C₁₋₃alkyl,
C₁₋₃alkoxy and trifluoromethyl;
W is selected from:
hydrogen and C₁₋₆alkyl, which is unsubstituted or substituted with 1-3 substituents where the substituents are independently selected from: halo, C₁₋₃alkoxy and trifluoromethyl;
Z is selected from:
C, N, and —O—, wherein when Z is N, then R⁴ is absent, and when W is —O—, then both R³ and R⁴ are absent;
n is an integer selected from 0, 1, 2, 3 and 4;
n is an integer selected from 1, 2, 3 and 4;
R¹ is selected from:
hydrogen, —C₀₋₆alkyl-, —(C₀₋₆alkyl)-alkenyl-,
—(C₀₋₆alkyl)-C₃₋₆cycloalkyl, —(C₀₋₆alkyl)-phenyl,
and —(C₀₋₆alkyl)-heterocycle,
where the alkyl is unsubstituted or substituted with 1-7 substituents where the substituents are independently selected from:
(a) halo,
(b) hydroxy,
(c) —O—C₁₋₃alkyl,
(d) trifluoromethyl, and
(e) —C₁₋₃alkyl,
and where the phenyl and the heterocycle is unsubstituted or substituted with 1-5 substituents where the substituents are independently selected from:
(a) halo,
(b) hydroxy; alkoxy
(c) amino; acylamino;sulfonylamino; alkoxycarbonylamino
(d) carboxylic acid; carbamide; sulfonamide
or wherein W and R¹ may be joined together to form a ring by a group selected from:
—(C₁₋₆alkyl)-, —C₀₋₆allyl-Y—(C₁₋₆alkyl)-, and
—(C₀₋₆alkyl)-Y—(C₀₋₆alkyl)-(C₃₋₇cycloalkyl)-(C₀₋₆alkyl),
where Y is selected from:
a single bond, —O—, —S—, —SO—, —SO₂—, and —NR¹⁰—,
and where the alkyl and the cycloalkyl are unsubstituted or substituted with 1-7 substituents where the substituents are independently selected from:
(a) halo,
(b) hydroxy,
(c) —O—C₁₋₃alkyl, and
(d) trifluoromethyl,
(e) C₁₋₃alkyl,
(f) —O—C₁₋₃alkyl,
(g) —CO₂R⁹, wherein R⁹ is independently selected from: hydrogen, C₁₋₆ alkyl, C₅₋₆ cycloalkyl, benzyl or phenyl, which is unsubstituted or substituted with 1-3 substituents where the substituents are independently selected from: halo, C₁₋₃alkyl, C₁₋₃alkoxy and trifluoromethyl,
(h) —CN,
(i) —NR⁹R¹⁰,
(j) —NR⁹COR¹⁰,
(k) —NR⁹SO₂R¹⁰, and
(l) —CONR⁹R¹⁰;
R² is selected from:
(C₀₋₆alkyl)-phenyl and (C₀₋₆alkyl)-heterocycle,
where the alkyl is unsubstituted or substituted with 1-7 substituents where the substituents are independently selected from:
(a) halo,
(b) hydroxy,
(c) —O—C₁₋₃alkyl,
(d) trifluoromethyl, and
(e) —C₁₋₃alkyl,
and where the phenyl and the heterocycle is unsubstituted or substituted with 1-5 substituents where the substituents are independently selected from:
(a) halo,
(b) trifluoromethyl,
(c) trifluoromethoxy,
(d) hydroxy,
(e) C₁₋₆alkyl,
(f) C₃₋₇cycloalkyl,
(g) —O—C₁₋₆alkyl,
(h) —O—C₃₋₇cycloalkyl,
(i) —SCF₃,
(j) —S—C₁₋₆alkyl,
(k) —SO₂-C₁₋₆alkyl,
(l) phenyl,
(m) heterocycle,
(n) —CO₂R⁹,
(o) —CN,
(p) —NR⁹R¹⁰,
(q) —NR⁹—SO₂—R¹⁰,
(r) —SO₂—NR⁹R¹⁰, and
(s) —CONR⁹R¹⁰;
R³ is —(C₀₋₆alkyl)-phenyl,
where the alkyl is unsubstituted or substituted with 1-5 substituents where the substituents are independently selected from:
(a) halo,
(b) hydroxy,
(c) —O—C₁₋₃alkyl, and
(d) trifluoromethyl,
and where the phenyl is unsubstituted or substituted with 1-5 substituents where the substituents are independently selected from:
(a) halo,
(b) trifluoromethyl
(c) hydroxy,
(d) C₁₋₃alkyl,
(e) —O—C₁₋₃alkyl,
(f) —CO₂R⁹,
(g) —CN,
(h) —NR⁹R¹⁰, and
(i) —CONR⁹R¹⁰;
R⁴ is selected from:
(a) hydrogen,
(b) hydroxy,
(c) C₁₋₆alkyl,
(d) C₁₋₆alkyl-hydroxy,
(e) —O—C₁₋₃alkyl,
(f) —CO₂R⁹,
(g) —CONR⁹R¹⁰, and
(h) —CN;
or where R³ and R⁴ may be joined together to form a ring which is selected from:
(a) 1H-indene,
(b) 2,3-dihydro-1H-indene,
(c) 2,3-dihydro-benzofuran,
(d) 1,3-dihydro-isobenzofuran,
(e) 2,3-dihydro-benzothiofuran, and
(f) 1,3-dihydro-isobenzothiofuran,
or where R³ and R⁵ or R⁴ and R⁶ may be joined together to form a ring which is phenyl, wherein the ring is unsubstituted or substituted with 1-7 substituents where the substituents are independently selected from:
(a) halo,
(b) trifluoromethyl,
(c) hydroxy,
(d) C₁₋₃alkyl,
(e) —O—C₁₋₃alkyl,
(f) —CO₂R⁹,
(g) —CN,
(h) —NR⁹R¹⁰, and
(i) —CONR⁹R¹⁰;
R⁵ and R⁶ are independently selected from:
(a) hydrogen,
(b) hydroxy,
(c) C₁₋₆alkyl,
(d) C₁₋₆alkyl-hydroxy,
(e) —O—C₁₋₃alkyl,
(f) oxo, and
(g) halo;
and pharmaceutically acceptable salts thereof and individual diastereomers thereof.

2. The compound of claim 1 of the formula Ia: 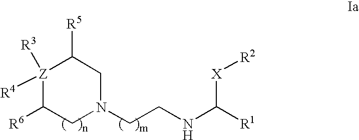
and pharmaceutically acceptable salts and individual diastereomers thereof.

3. The compound of claim 1 of the formula Ib: 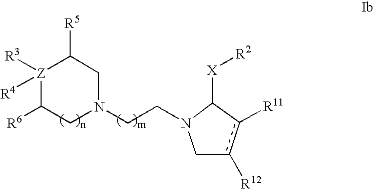
wherein:
the dashed line represents a single or a double bond;
R¹¹ is selected from:
(a) hydrogen
(b) C₁₋₆alkyl
(c) hydroxy,
(d) —O—C₁₋₃alkyl
(e) -Phenyl and heterocycle,
(f) —CO₂R⁹,
(g) —CN,
(h) —NR⁹R¹⁰, and
(i) —CONR⁹R¹⁰;
R¹² is selected from:
(a) hydrogen,
(b) hydroxy,
(c) C₁₋₆alkyl,
(d) C₁₋₆alkyl-hydroxy,
(e) —O—C₁₋₃alkyl,
(f) —CO₂R⁹,
(g) —CONR⁹R¹⁰, and
(h) —CN;
or where R¹¹ and R¹² may be joined together to form a ring which is selected from:
(a) benzene,
(b) furan,
(c) thiophene,
(d) thiazole,
(e) C₃₋₆cycloalkyl
wherein the ring is unsubstituted or substituted with 1-7 substituents where the substituents are independently selected from:
(a) halo,
(b) trifluoromethyl,
(c) hydroxy,
(d) C₁₋₃alkyl,
(e) —O—C₁₋₃alkyl,
(f) —CO₂R⁹,
(g) —CN,
(h) —NR⁹R¹⁰, and
(i) —CONR⁹R¹⁰;
and pharmaceutically acceptable salts and individual diastereomers thereof.

4. The compound of claim 3 of the formula Id: 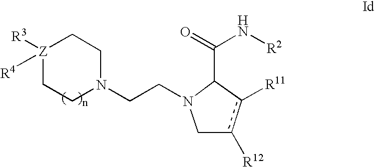
and pharmaceutically acceptable salts and individual diastereomers thereof.

5. The compound of claim 3 of the formula Ie: 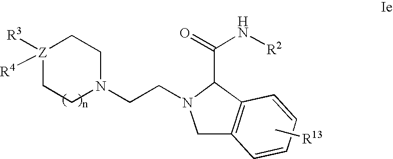
wherein R¹³ is independently selected from:
(a) hydrogen,
(b) halo,
(c) trifluoromethyl,
(d) fused C₁₋₃cycloalkyl
(e) C₁₋₃alkyl,
(f) —O—C₁₋₃alkyl,
(g) —CO₂H,
(h) —CO₂C₁₋₃alkyl, and
(i) —CN;
and pharmaceutically acceptable salts and individual diastereomers thereof.

6. The compound of claim 3 of the formula If: 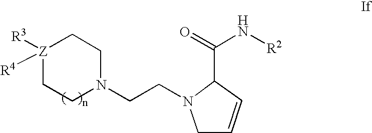
and pharmaceutically acceptable salts and individual diastereomers thereof.

7. The compound of claim 1 of the formula II: 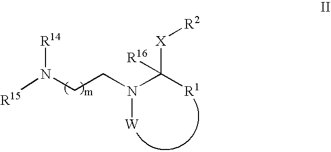
wherein R¹⁴, R¹⁵, R¹⁶ are independently selected from:
(a) hydrogen,
(b) —C₁₋₆alkyl
(c) —C₁₋₆cycloalkyl
(d) —C₁₋₆alkyl-phenyl
(e) —C₁₋₆alkyl-heterocylcle
(f) —C₁₋₆alkyl-C₃₋₆cycloalkyl
(g) —C₁₋₆alkyl O—C₁₋₆alkyl,
and pharmaceutically acceptable salts and individual diastereomers thereof.

8. The compound of claim 1 of the formula IIa: 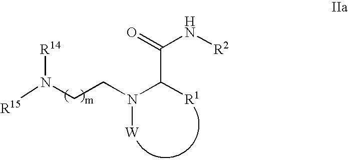
and pharmaceutically acceptable salts and individual diastereomers thereof.

9. The compound of claim 1 of the formula IIb: 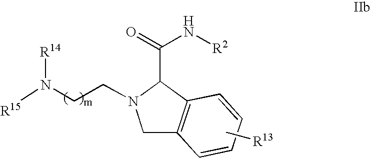
and pharmaceutically acceptable salts and individual diastereomers thereof.

10. The compound of claim 1 of the formula IIc: 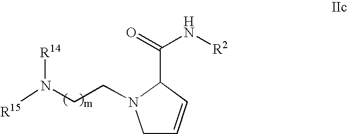
and pharmaceutically acceptable salts and individual diastercomers thereof.

11. The compound of claim 1 of the formula: 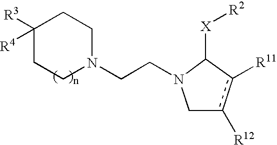
wherein:
the dashed line represents a single or a double bond,
R¹¹ and R¹² are hydrogen or where R¹¹ and R¹² may be joined together to form a ring which is selected from:
(a) benzene,
(b) heterocycle
(c) C₃₋₆cycloalkyl
and pharmaceutically acceptable salts and individual diastereomers thereof.

12. The compound of claim 1 of the formula: 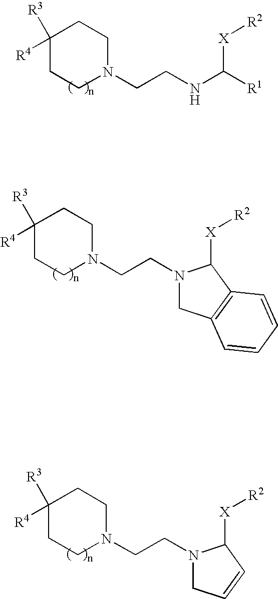
and pharmaceutically acceptable salts and individual diastereomers thereof.

13. The compound of claim 1 wherein W is hydrogen or —CH₂—.

14. The compound of claim 1 wherein X is —CONH—, phenyl or heterocycle.

15. The compound of claim 1 wherein Z is —C— or —N—.

16. The compound of claim 1 wherein n is 0 and 1.

17. The compound of claim 1 wherein m is 1.

18. The compound of claim 1 wherein heterocycle is selected from: furanyl, imidazolyl, oxadiazolyl, oxazolyl, pyrazolyl, pyrazinyl, pyridyl, pyridazinyl, pyrimidyl, pyrrolyl, thiadiazolyl, thiazolyl, thienyl, and triazolyl, and N-oxides thereof.

19. The compound of claim 1 wherein —C₁₋₆alkyl, —C₀₋₆alkyl-O—C₁₋₆alkyl-, —C₀₋₆alkyl-S—C₁₋₆alkyl-, and —(C₀₋₆allyl)-(C₃₋₇cycloalkyl)-(C₀₋₆alkyl),
where the alkyl and the cycloalkyl are unsubstituted or substituted with 1-7 substituents where the substituents are independently selected from:
(a) halo,
(b) hydroxy,
(c) —O—C₁₋₃alkyl,
(d) trifluoromethyl,
(f) C₁₋₃alkyl,
(g) —O—C₁₋₃alkyl,
(h) —CO₂R⁹, wherein R⁹ is independently selected from: hydrogen, C₁₋₆ alkyl, C₅₋₆ cycloalkyl, benzyl or phenyl, which is unsubstituted or substituted with 1-3 substituents where the substituents are independently selected from: halo, C₁₋₃alkyl, C₁₋₃alkoxy and trifluoromethyl,
(i) —CN,
(j) —NR⁹R¹⁰, and
(k) —CONR⁹R¹⁰.

20. The compound of claim 1 wherein R¹ is selected from:
(1) —C₁₋₆alkyl, which is unsubstituted or substituted with 1-6 substituents where the substituents are independently selected from:
(a) halo,
(b) hydroxy,
(c) —O—C₁₋₃alkyl, and
(d) trifluoromethyl,
(2) —C₀₋₆alkyl-O—C₁₋₆alkyl-, which is unsubstituted or substituted with 1-6 substituents where the substituents are independently selected from:
(a) halo, and
(b) trifluoromethyl,
(3) —C₀₋₆alkyl-S—C₁₋₆alkyl-, which is unsubstituted or substituted with 1-6 substituents where the substituents are independently selected from:
(a) halo, and
(b) trifluoromethyl,
(4) —(C3 5cycloalkyl)-(C₀₋₆alkyl), which is unsubstituted or substituted with 1-7 substituents where the substituents are independently selected from:
(a) halo,
(b) hydroxy,
(c) —O—C₁₋₃alkyl, and
(d) trifluoromethyl.

21. The compound of claim 1 wherein R¹ is selected from:
(1) —CH₃,
(2) —CH₂CH₃,
(3) —CH(CH₃)₂,
(4) —CH₂CH₂CH₃,
(5) —CH₂CH(CH₃)₂,
(6) -cyclopropyl,
(7) -cyclobutyl,
(8) -cyclopentyl,
(9) —CH₂-cyclopropyl,
(10) —CH₂-cyclobutyl,
(11) —CH₂-cyclopentyl,
(12) —CH₂OH,
(13) —C(CH₃)2(OH),
(14) —C(CH₂OH)(CH₃)₂,
(15) —(OH)cyclobutyl,
(16) —(OH)cyclopentyl,
(17) —C(CH₃)₂(NHCOCH₃),
(18) —C(CO₂H)(CH₃)₂,
(19) —O—CH₃,
(20) —O—cyclopentyl,
(21) —O—CH(CH₃)₂,
(22) —S—CH₃,
(23) —S—CF₃,
(24) —SO₂—CH₃,
(25) —S—CH(CH₃)₂,
(26) —SO₂—CH(CH₃)₂, and
(27) —NH—SO₂—CH₃.

22. The compound of claim 1 wherein R² is selected from —(C₀₋₄alkyl)-phenyl and —(C₀₋₄alkyl)-heterocycle,
where heterocycle is selected from:
furanyl, imidazolyl, oxadiazolyl, oxazolyl, pyrazolyl, pyrazinyl, pyridyl, pyridazinyl, pyrimidyl, pyrrolyl, thiadiazolyl, thiazolyl, thienyl, and triazolyl and N-oxides thereof,
where the alkyl is unsubstituted or substituted with 1-7 substituents where the substituents are independently selected from:
(a) halo,
(b) hydroxy,
(c) —O—C₁₋₃alkyl and
(d) trifluoromethyl,
and where the phenyl or heterocycle is unsubstituted or substituted with 1-5 substituents where the substituents are independently selected from:
(a) halo,
(b) trifluoromethyl,
(c) trifluoromethoxy,
(d) hydroxy,
(e) C₁₋₃alkyl,
(f) —O—C₁₋₃alkyl
(g) —CO₂R⁹,
(h) —S—C₁₋₃alkyl
(i) —SO₂—C₁₋₃alkyl,
(j) —SCF₃,
(k) —CO₂R⁹,
(l) —NR⁹R¹⁰,
(m) —NR⁹—SO₂R¹⁰,
(n) —SO₂—NR⁹R¹⁰, and
(o) —CONR⁹R¹⁰.

23. The compound of claim 1 wherein R² is selected from —(C₀₋₄alkyl)-phenyl and —(C₀₋₄alkyl)-heterocycle,
where heterocycle is selected from: pyridyl, pyridazinyl, and N-oxides thereof,
where the alkyl is unsubstituted or substituted with 1-7 substituents where the substituents are independently selected from:
(a) halo,
(b) hydroxy,
(c) —O—C₁₋₃alkyl, and
(d) trifluoromethyl,
and where the phenyl or heterocycle is unsubstituted or substituted with 1-3 substituents where the substituents are independently selected from:
(a) halo,
(b) trifluoromethyl,
(c) trifluoromethoxy,
(d) hydroxy,
(e) C₁₋₃alkyl,
(f) —O—C₁₋₃alkyl,
(g) —CO₂—C₁₋₃alkyl,
(h) —CO₂H,
(i) —SC₁₋₃alkyl,
(j) —SO₂—C₁₋₃alkyl,
(k) —SCF₃,
(l) —NH₂,
(m) —NH-SO₂-C₁₋₃alkyl, and
(n) —SO₂—NH₂.

24. The compound of claim 1 wherein R² is selected from —CH₂-phenyl and —CH₂-heterocycle,
where heterocycle is selected from: pyridyl, pyridazinyl, and N-oxides thereof,
and where the phenyl or heterocycle is unsubstituted or substituted with 1-3 substituents where the substituents are independently selected from:
(a) halo,
(b) trifluoromethyl,
(c) trifluoromethoxy,
(d) hydroxy,
(e) C₁₋₃alkyl
(f) —O—C₁₋₃alkyl,
(g) —CO₂-C₁₋₃alkyl,
(h) —CO₂H,
(i) —S—C₁₋₃alkyl,
(j) —SO₂—C₁₋₃alkyl,
(k) —SCF₃,
(l) —NH₂,
(m) —NH—SO₂—C₁₋₃alkyl and
(n) —SO₂—NH₂.

25. The compound of claim 1 wherein R² is selected from:
(1) —CH₂-(phenyl),
(2) —CH₂-(4-bromophenyl),
(3) —CH₂-(3-chlorophenyl),
(4) —CH₂-(3,5-difluorophenyl),
(5) —CH₂-((2-trifluoromethyl)phenyl),
(6) —CH₂-((3-trifluoromethyl)phenyl),
(7) —CH₂-((4trifluoromethyl)phenyl),
(8) —CH₂-((3-trifluoromethoxy)phenyl),
(9) —CH₂-((3-trifluoromethylthio)phenyl),
(10) —CH₂-((3-trifluoromethoxy-5-thiomethyl)phenyl),
(11) —CH₂-((3-trifluoromethoxy-5-methoxy)phenyl),
(12) —CH₂-((3-trifluoromethoxy-5-methanesulfonyl)phenyl),
(13) —CH₂-((3-trifluoromethoxy-5-amino)phenyl),
(14) —CH₂-((3-trifluoromethoxy-5-aminomethanesulfonyl)phenyl),
(15) —CH₂-((3-trifluoromethoxy-5-sulfonylamino)phenyl),
(16) —CH₂-((3,5-bis-trifluoromethyl)phenyl),
(17) —CH₂-((3-fluoro-5-trifluoromethyl)phenyl),
(18) —CH(CH₃)-((3,5-bis-trifluoromethyl)phenyl),
(19) —C(CH₃)₂-((3,5-bis-trifluoromethyl)phenyl),
(20) —CH₂-(4-(2-trifluoromethylpyridyl),
(21) —CH₂-(5-(3-trifluoromethyl)pyridyl),
(22) —CH₂-(5-(3-tifluoromethyl)pyridazinyl),
(23) —CH₂-(4-(2-trifluoromethylpyridyl-N-oxide), and
(24) —CH₂-(S-(3-trifluoromethyl)pyridyl-N-oxide).

26. The compound of claim 1 wherein R³ is hydrogen and phenyl
where the phenyl is unsubstituted or substituted with 1-5 substituents where the substituents are independently selected from:
(a) halo,
(b) trifluoromethyl,
(c) hydroxy,
(d) C₁₋₃alkyl,
(e) —O—C₁₋₃alkyl,
(f) —CO₂R⁹,
(g) —CN,
(h) —NR⁹R¹⁰, and
(i) —CONR⁹R¹⁰.

27. The compound of claim I wherein R³ is hydrogen and phenyl, where the phenyl is unsubstituted or substituted with 1-3 substituents where the substituents are independently selected from:
(a) halo,
(c) hydroxy,
(d) C₁₋₃alkyl,
(e) —O—C₁₋₃alkyl, and
(f) —CO₂R⁹.

28. The compound of claim 1 wherein R³ is phenyl, or para-fluorophenyl.

29. The compound of claim 1 wherein R⁴ is selected from:
(a) hydrogen,
(b) hydroxy,
(c) —CO₂H,
(d) —CO₂C₁₋₆alkyl, and
(e) —CN.

30. The compound of claim 1 wherein R⁵ and R⁶ are independently selected from:
(a) hydrogen,
(b) hydroxy,
(c) —CH₃,
(d) —O—CH₃, and
(e) oxo.

31. A compound which is selected from the group consisting of the title compounds of the Examples, and pharmaceutically acceptable salts and individual diastereomers thereof.

32. A pharmaceutical composition which comprises an inert carrier and a compound of claim 1.

33. A method for modulation of chemokine receptor activity in a mammal in need thereof which comprises the administration of an effective amount of the compound of claim 1.

34. A method for treating, ameliorating or controlling an inflammatory or immunoregulatory disorder or disease which comprises administering to a patient in need thereof an effective amount of the compound of claim 1.

35. A method for reducing the risk of an inflammatory or immunoregulatory disorder or disease which comprises administering to a patient in need thereof an effective amount of the compound of claim 1.

36. A method for treating, ameliorating or controlling rheumatoid arthritis which comprises administering to a patient in need thereof an effective amount of the compound of claim 1.
